(19) United States
(12) Patent Application Publication
(10) Pub. No.: US 2010/0081849 A1
(43) Pub. Date: Apr. 1, 2010

(54) REACTION SYSTEM

(75) Inventors: Anthony Frederick Masters, Glebe (AU); Alessandro Fois, Eartlwood (AU); Thomas Maschmeyer, Lindfield (AU)

Correspondence Address: WOLF GREENFIELD & SACKS, P.C., 600 ATLANTIC AVENUE, BOSTON, MA, 02210-2206, US

(73) Assignee: THE UNIVERSITY OF SYDNEY

(21) Appl. No.: 12/310,294

(22) PCT Filed: Aug. 23, 2007
(86) PCT No.: PCT/AU2007/001212

§ 371 Date: Oct. 21, 2009

Related U.S. Application Data
(60) Provisional application No. 60/840,009, filed on Aug. 25, 2006.

(51) Int. Cl. C07C 29/92; B01J 8/02

(52) U.S. Cl. 568/808; 568/810; 422/191

(57) ABSTRACT

The invention provides a reaction system comprising: a first reaction promoter capable of converting a first substrate into a first substance; and a plurality of microcapsules, each of said microcapsules comprising a second reaction promoter encapsulated within an encapsulant, said second reaction promoter being capable of converting a second substrate into a second substance. The second substrate is capable of passing through the encapsulant to contact the second reaction promoter and the second substance is capable of passing out of the microcapsules through the encapsulant. In the reaction system, either (a) the first substance is, or is capable of being converted into, the second substrate and, in operation, the conversion of the second substrate by the second reaction promoter occurs to a greater extent than the conversion of the first substrate by the second reaction promoter and the conversion of the second substance by the first reaction promoter is low, or (b) the second substance is, or is capable of being converted into, the first substrate and, in operation, the conversion of the first substrate by the first reaction promoter occurs to a greater extent than the conversion of the second substrate by the first reaction promoter and the conversion of the first substance in the microcapsules is low.

### TECHNICAL FIELD

The present invention relates to a reaction system having two reaction promotors, at least one of which is encapsulated within microcapsules.

### BACKGROUND OF THE INVENTION

The ability to combine multiple, mutually-interfering catalysts within a single reaction system has long been a subject of investigation, particularly in the area of Dynamic Kinetic Resolution. Previous solutions to the problem have included membrane reactors, biphasic systems and sequential addition of catalysts, but the complicated nature of these approaches is a significant disadvantage.

Given the ongoing search for more efficient catalysts and a concomitant appreciation of the elegance of natural catalytic systems, it is of little surprise that extensive research has been undertaken into the mimicry of natural catalysis. The localization of reactions within cells has inspired a wide variety of artificial nanoreactors. These nanoreactors have been prepared generally by using supramolecular assembly, self-assembly in particular. Recently this supramolecular form of catalysis has enjoyed considerable interest as it has application to many different catalytic reactions. These systems typically consist of a capsule, of nanometer or micrometer dimensions, acting as a catalyst or containing a catalytic material, and within which the reaction proceeds.

Nanoreactor catalysis can proceed by assembly and disassembly of the capsules to allow product release and further substrate conversion. Nanoreactors that function via permeation of substrate through the capsule walls to react with an encapsulated catalyst can also be prepared.

These capsules can be tailored to provide regio- or stereoselectivity in product formation through geometric constriction of the encapsulated substrates. A variety of asymmetric capsules has been prepared, however these have provided generally poor enantioselectivity and have proven difficult to synthesize. Moreover, selective permeability of the capsule membranes could play a key role in a selective catalytic system by allowing only a certain substrate to access the catalyst or a specific product to escape the capsule. These systems thus offer a potentially powerful new approach to more selective and recoverable catalysts.

### OBJECT OF THE INVENTION

It is the object of the present invention to substantially overcome or at least ameliorate one or more of the above disadvantages.

### SUMMARY OF THE INVENTION

In a first aspect of the invention there is provided a reaction system comprising:
a first reaction promoter capable of converting a first substrate into a first substance; and
a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant;
   whereby either (a) the first substance is, or is capable of being converted into, the second substrate and, in operation, the conversion of the second substrate by the second reaction promoter occurs to a greater extent than the conversion of the first substrate by the second reaction promoter and the conversion of the second substance by the first reaction promoter is low, optionally negligible, optionally zero, or (b) the second substance is, or is capable of being converted into, the first substrate and, in operation, the conversion of the first substrate by the first reaction promoter occurs to a greater extent than the conversion of the second substrate by the first reaction promoter and the conversion of the first substance in the microcapsules is low, optionally negligible, optionally zero.

Thus in case (a), the overall reaction promoted by the first reaction promoter and the microcapsules may be:

S1→(P1)→S2→(MC)→C2

or

S1→(P1)→C1→S2→(MC)→C2

and in case (b), the overall reaction promoted by the first reaction promoter and the microcapsules may be:

S2→(MC)→S1→(P1)→C1

or

S2→(MC)→C2→S1→(P1)→C1

where S1 and S2 are the first and second substrates respectively, C1 and C2 are the first and second substances respectively, P1 is the first reaction promoter and MC is the microcapsules, which comprise the second reaction promoter encapsulated within an encapsulant. In these equations, the symbolism →(X)→ represents a reaction promoted by species X.

In relation to equation S1→(P1)→S2→(MC)→C2, a first substrate S1 is converted by means of a first reaction promoter P1 into a second substrate S2. The second substrate penetrates into microcapsules MC and is converted to the second substance C2 by means of the second reaction promoter, which is encapsulated in the microcapsules. The second substance then migrates out of the microcapsules. The second substance may be the overall product of the reaction sequence. In relation to equation S1→(P1)→C1→S2→(MC)→C2, the first substrate S1 is converted by means of the first reaction promoter into a first substance C1, which then converts into the second substrate S2. The second substrate then penetrates into the microcapsules MC and is converted to the second to substance C2 by means of the second reaction promoter, which is encapsulated in the microcapsules. The second substance then migrates out of the microcapsules.

In relation to equation S2→(MC)→S1→(P1)→C1, a second substrate S2 penetrates into microcapsules MC and is converted to a first substrate S1 by means of a second reaction promoter, which is encapsulated in the microcapsules. The first substrate is then migrates out of the microcapsules and is converted by means of a first reaction promoter P1 into the first substance C1, which may be the overall product of the reaction. In relation to equation S2→(MC)→C2→S1→(P1)→C1, the second substrate S2 penetrates into microcapsules MC and is converted to a second substance C2 by means of a second reaction promoter, which is encapsulated in the microcapsules. The second substance either migrates out of the microcapsules and then converts to the first substrate S1, or else converts to the first substrate inside the microcapsules and the first substrate migrates out of the microcapsules. The first substrate is then converted by means of the first reaction promoter P1 into the first substance C1, which may be the overall product of the reaction.

In case (a) the rate of conversion of the second substance by the first reaction promoter may be sufficiently low as to allow either separation of the second substance with acceptable yield and/or purity or further reaction of the second substance to a product having acceptable yield and/or purity. In this case the conversion of the second substance, if it occurs, may be into one or more undesirable by-products. In case (a) also, the first reaction promoter may be capable of interconverting the first substrate and the second substrate, i.e. it may be capable of converting the first substrate into the second substrate and of converting the second substrate into the first substrate. In this case, the microcapsules may be capable of selectively converting the second substrate to the second substance, either because the second reaction promoter is capable of selectively converting the second substrate to the second substance or because the encapsulant is capable of selectively transmitting the second substrate. Similarly, in case (b) the rate of conversion of the first substance by the second reaction promoter may be sufficiently low as to allow either separation of the first substance with acceptable yield and/or purity or further reaction to a product having acceptable yield and/or purity. In this case the conversion of the first substance, if it occurs, may be into one or more undesirable by-products. In case (b) also, the second reaction promoter may be capable of interconverting the first substrate and the second substrate, i.e. it may be capable of converting the first substrate into the second substrate and of converting the second substrate into the first substrate. In this case to the first reaction promoter may be capable of selectively converting the first substrate to the first substance.

The first reaction promoter and the second reaction promoter may, independently, comprise a catalyst or a reagent or a reagent combined with a catalyst, or may comprise more than one catalyst and/or reagent. In the present specification, “converting” a material may refer to catalysing reaction of the material or to reacting with the material or a combination of catalysing and reacting.

The first substance (in case b above) or the second substance (in case a above) may be a final product. Alternatively, the reaction system may optionally comprise further reaction promoters (each independently being a catalyst or a reagent or a reagent combined with a catalyst) which may be, independently, encapsulated or not encapsulated. One or more of the further reaction promoters may be capable of converting the second substance (in case a above) or converting the first substance (in case b above). In this case, the rates of conversion of a final product in the microcapsules and by the first and further reaction promoters may be low, optionally negligible, optionally zero. These rates of conversion may be sufficiently low as to allow separation of the final product in acceptable yield and purity.

The first reaction promoter may be encapsulated or not encapsulated. The further reaction promoters (if present) may each, optionally and independently, comprise a reagent or a catalyst or a reagent combined with a catalyst. The further reaction promoters (if present) may each, independently, be encapsulated or not encapsulated. An encapsulated reaction promoter may be encapsulated in the same microcapsules as the second reaction promoter, or as any other encapsulated reaction promoter, or in different microcapsules. The encapsulant for any encapsulated reaction promoter may be the same as or different to the encapsulant for any other encapsulated reaction promoter. For any encapsulated reaction promoter, the encapsulant in which it is encapsulated may be at least partially permeable to a desired substrate and to a desired product of reaction of said desired substrate promoted by the encapsulated reaction promoter. The encapsulant may be substantially impermeable to the encapsulated reaction promoter, and may be substantially impermeable to a substance which is capable of deactivating the reaction promoter.

The conversion of the first substance (case b above) or the second substance (case a above) by the second reaction promoter may be low either because the rate of conversion of said substance by the second reaction promoter is low, or because said substance passes slowly through the encapsulant or is incapable of passing therethrough. Said rate of conversion may be zero, substantially zero or negligible. This may be because the rate of conversion of said substance by the second reaction promoter is zero, substantially zero or negligible, or it may be because said substance passes through the encapsulant to contact the second reaction promoter at a rate which is zero, substantially zero or negligible.

The reaction system may additionally comprise a separator for separating a product from the first reaction promoter and from the microcapsules. It may also comprise a purifier for purifying the separated product. The product may comprise the first substance (particularly in case b) or the second substance (particularly in case a), or may comprise a product derived from either the first or second substance by one or more reactions promoted by further reaction promoters (if present).

The conversion of a substrate to the corresponding substance may be a selective conversion. The conversion of the first substrate to the first substance may be a selective conversion, i.e. the first reaction promoter may be a selective reaction promoter, and may be incapable of promoting conversion of the second substrate, or may promote conversion of the second substrate at a slower rate than of the first substrate. The conversion of the second substrate to the second substance may be a selective conversion, i.e. the second reaction promoter may be incapable of promoting conversion of the first substrate, or may promote conversion of the first substrate at a slower rate than of the second substrate, either because the second reaction promoter is a selective reaction promoter or because the first substrate is incapable of passing through the encapsulant to contact the second reaction promoter, or passes therethrough at a slow rate, or at a slower rate than that of the second substrate.

The encapsulant may comprise a polymer. It may be selectively permeable. It may comprise a polyelectrolyte. It may comprise more than one layer. The, or each, layer may be between about 2 and about 50 nm thick. The encapsulant may comprise an electrically charged polymeric layer. The encapsulant may comprise at least one positively charged polymeric layer and at least one negatively charged polymeric layer. If more than one of either a positively charged or a negatively charged polymeric layer, or both, are present, then the positively charged and negatively charged layers may alternate. The innermost layer and, independently, the outermost layer, may be a negatively charged polymeric layer or a positively charged polymeric layer, or may be some other type of layer, for example an uncharged layer or a charged non-polymeric layer.

The first and second reaction promoters may be capable of interacting (e.g. reacting) so as to deactivate one or both thereof, but in the reaction system of the present invention to may be at least partially prevented from doing so due to encapsulation of the second reaction promoter. In this context, deactivating refers to converting the reaction promoter into a form in which it is incapable of promoting the reaction which it is capable of promoting in the undeactivated form. The encapsulant may be impermeable to the first reaction promoter and to the second reaction promoter.

The first reaction promoter may be dispersed, suspended, dissolved or otherwise distributed within a reaction medium, e.g. a solvent. The microcapsules may be dispersed, suspended or otherwise distributed within the reaction medium. Microcapsules may be nanocapsules or nanoreactors. The microcapsules may have a mean diameter of between about 0.2 and about 10 microns.

The microcapsules may comprise an energy absorber for absorbing energy (e.g. radiation), optionally for converting the energy to a form in which the energy can be transferred to the second substrate and/or to the second reaction promoter in order to promote the reaction promoted by the second reaction promoter. In some embodiments, the energy absorber is a radiation absorber. It may for example be capable of absorbing radiation so as to raise the temperature locally within the capsules or otherwise distribute the radiation, so as to accelerate, or otherwise influence, the conversion of the second substrate.

In an embodiment there is provided a reaction system comprising:
a first catalyst capable of converting a first substrate to a product; and
a plurality of microcapsules, each of said microcapsules comprising a second catalyst encapsulated within an encapsulant, said second catalyst being capable of converting a second substrate to the first substrate, wherein the second substrate is capable of passing through the encapsulant to contact the second catalyst and the first substrate is capable of passing out of the microcapsules through the encapsulant;
   whereby, in operation, the conversion of the first substrate by the first catalyst is greater than the conversion of the second substrate by the first catalyst, and the conversion of the product to a by-product in the microcapsules is low, optionally negligible, optionally zero.

In another embodiment there is provided a reaction system for selective reaction of a first substrate in the presence of a second substrate, said system comprising:
a first catalyst capable of converting the first substrate to a product at a rate greater than it converts the second substrate to a by-product; and
a plurality of microcapsules, each of said microcapsules comprising a second catalyst encapsulated within an encapsulant, said second catalyst being capable of converting the second substrate to the first substrate, wherein the second substrate is capable of passing through the encapsulant to contact the second catalyst and the first substrate is capable of passing out of the microcapsules through the encapsulant;
   whereby, in operation, the conversion of the product, optionally to the by-product, in the microcapsules is low, optionally negligible, optionally zero.

In another embodiment there is provided a reaction system for Dynamic Kinetic Resolution of a racemic alcohol comprising:
a first catalyst capable of esterifying a first optical isomer of the alcohol to form a first optical isomer of an ester of the alcohol at a rate greater than it esterifies a second optical isomer of the alcohol; and
a plurality of microcapsules, each of said microcapsules comprising a second catalyst encapsulated within an encapsulant, said second catalyst being capable of racemising the second optical isomer of the alcohol, wherein the second optical isomer of the alcohol is capable of passing through the encapsulant to contact the second catalyst and the first optical isomer of the alcohol is capable of passing out of the microcapsules through the encapsulant;
   whereby the rate of racemisation of the first optical isomer of the ester of the alcohol in the microcapsules is low, optionally negligible, optionally zero.

The first catalyst may be a chiral catalyst, for example an enzyme. The second catalyst may be an acidic catalyst, for example a zeolite. The enzyme and the zeolite may be capable of interacting so as to deactivate the enzyme, but in the reaction system of the present invention may be at least partially prevented from doing so due to encapsulation of the zeolite.

In a second aspect of the invention there is provided a reaction system comprising:
a first reaction promoter capable of converting a first substrate into a first substance; and
a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant;
   whereby the first and second reaction promoters are capable of interacting so as to deactivate one or both thereof, but in the reaction system are at least partially prevented from doing so due to encapsulation of the second reaction promoter.

The encapsulant may be impermeable to the first reaction promoter and to the second reaction promoter. In an embodiment, either (a) the first substance is the second substrate, or (b) the second substance is the first substrate.

In a third aspect of the invention there is provided a method for conducting a reaction comprising:
providing a reaction system according to the first aspect or the second aspect of the invention; and
adding either the first substrate or the second substrate or both the first substrate and the second substrate to the reaction system;
   whereby either (a) the first substrate is converted either directly or indirectly to the second substrate and the second substrate is converted to the second substance, or (b) the second substrate is converted either directly or indirectly to the first substrate and the first substrate is converted to the first substance.

There is therefore provided a method for conducting a reaction comprising:
providing a reaction system comprising (i) a first reaction promoter capable of converting a first substrate into a first substance; and (ii) a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant; and
adding either the first substrate or the second substrate or both the first substrate and the second substrate to the reaction system;
   whereby either (a) the first substance is, or is capable of being converted into, the second substrate, such that the first substrate is converted either directly or indirectly to the second substrate and the second substrate is converted to the second substance, or (b) the second substance is, or is capable of being converted into, the first substrate, such that the second substrate is converted either directly or indirectly to the first substrate and the first substrate is converted to the first substance.

There is also provided a method for conducting a reaction comprising:
providing a reaction system comprising (i) a first reaction promoter capable of converting a first substrate into a first substance; and (ii) a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant; and
adding either the first substrate or the second substrate or both the first substrate and the second substrate to the reaction system; whereby the first and second reaction promoters are capable of interacting so as to deactivate one or both thereof, but in the reaction system are at least partially prevented from doing so due to encapsulation of the second reaction promoter.

In an embodiment, either (a) the first substance is the second substrate, or (b) the second substance is the first substrate.

In options (a), the first reaction promoter may interconvert the first and second substrates. In options (b) the second reaction promoter may interconvert the first and second substrates.

The first reaction promoter and the second reaction promoter may, independently, be a catalyst or a reagent or a combination of a catalyst and a reagent. The first substrate and the second substrate may be added together to the reaction system. In this case, in a separate step, the first substance may be subsequently converted into the first substrate (in option b above), or the second substance may be converted into the second substrate (in option a above), whereby this aspect provides a method for selectively converting one substrate into the other substrate.

The method may comprise separating the product from the reaction system. The separating may comprise filtering, centrifuging, membrane separation, settling, decanting, chromatographic separation (e.g. hplc, gc, gpc, sec, affinity chromatography, tlc) or some combination of two or more of these, and may additionally or alternatively comprise some other separation technique.

The method may comprise heating the reaction system and/or irradiating the reaction system with radiation of a wavelength capable of being absorbed by a component of the reaction system, by the first or second substrate or by more than one of these.

The method may also comprise reacting the product of the overall reaction. The reacting may convert the product into either the first or the second substrate, as noted above. In this case the method may represent a process for selectively converting one substrate into the other substrate.

In an embodiment there is provided a method for conducting a reaction comprising:
providing a reaction system comprising (i′) a first catalyst capable of converting a first substrate to a product; and (ii′) a plurality of microcapsules, each of said microcapsules comprising a second catalyst encapsulated within an encapsulant, said second catalyst being capable of converting a second substrate to the first substrate, wherein the second substrate is capable of passing through the encapsulant to contact the second catalyst and the first substrate is capable of passing out of the microcapsules through the encapsulant; whereby the conversion of the product to a by-product by the microcapsules is low, optionally negligible, optionally zero; and
adding either the first substrate or the second substrate or both the first substrate and the second substrate to the reaction system;
   whereby the second substrate is converted to the first substrate, optionally interconverted with the first substrate, said first substrate passing out of the microcapsules through the encapsulant and being converted to the product.

The first substrate and the second substrate may be added together to the reaction system. In this case, the product may be subsequently converted into the first substrate, whereby the embodiment provides a method for separating the first substrate from the second substrate.

In another embodiment there is provided a method for selective reaction of a first substrate in the presence of a second substrate, said method comprising:
providing a reaction system comprising (i″) a first catalyst capable of converting the first substrate to a product at a rate greater than it converts the second substrate to a by-product; and (ii″) a plurality of microcapsules, each of said microcapsules comprising a second catalyst encapsulated within an encapsulant, said second catalyst being capable of converting the second substrate to the first substrate, wherein the second substrate is capable of passing through the encapsulant to contact the second catalyst and the first substrate is capable of passing out of the microcapsules through the encapsulant; whereby the rate of conversion of the product, optionally to the by-product, in the microcapsules is low, optionally negligible, optionally zero; and
adding the first substrate and the second substrate to the reaction system;
   whereby the second substrate is converted to the first substrate, optionally interconverted with the substrate, and the first substrate is selectively converted to the product.

In an embodiment there is provided a method for Dynamic Kinetic Resolution of a racemic alcohol comprising:
providing a reaction system comprising (i′″) a first catalyst capable of esterifying a first optical isomer of the alcohol to form a first optical isomer of an ester of the alcohol at a rate greater than it esterifies a second optical isomer of the alcohol; and (ii′″) a plurality of microcapsules, each of said microcapsules comprising a second catalyst encapsulated within an encapsulant, said second catalyst being capable of racemising the second optical isomer of the alcohol, wherein the second optical isomer of the alcohol is capable of passing through the encapsulant to contact the second catalyst and the first optical isomer of the alcohol is capable of passing out of the microcapsules through the encapsulant; whereby the rate of racemisation of the first optical isomer of the ester of the alcohol in the microcapsules is low, optionally negligible, optionally zero; and
adding the racemic alcohol to the reaction system;
   whereby the first optical isomer of the alcohol is converted to the first optical isomer of the ester and the second optical isomer is racemised to form a mixture of the first and second optical isomers of the alcohol.

Both the first and second optical isomers of the alcohol may be capable of passing through the encapsulant. The microcapsules may be incapable of racemising the chiral ester, or the rate of racemisation of the chiral alcohol may be low or negligible, because the second catalyst is incapable of racemising the chiral ester or racemises it at a low or negligible rate, or because the chiral ester is incapable of passing through the encapsulant to contact the second catalyst or passes therethrough at a low or negligible rate.

The first catalyst may be a chiral catalyst, for example an enzyme. The second catalyst may be an acidic catalyst, for example a zeolite.

The method may additionally comprise one or more of the steps of:
separating the chiral ester from the reaction system;
purifying the chiral ester;
hydrolysing the chiral ester to produce the first optical isomer of the alcohol; and
purifying the first isomer of the alcohol.

In a fourth aspect of the invention there is provided a reactor comprising:
a reactor vessel; and
a reaction system according to the first aspect or the second aspect of the invention, said reaction system being disposed within the reactor vessel.
   The reaction system may additionally comprise a separator for separating a product from the first reaction promoter and from the microcapsules. It may also comprise a purifier for purifying the separated product. It may also comprise an addition port for adding substrate(s), reagent(s) and/or reaction medium to the reactor. It may also comprise a product port for removing product from the reactor.

In a fifth aspect of the invention there is provided a product produced by a reaction system according to the first aspect or the second aspect of the invention, or produced by the method of the third aspect of the invention. The product may be a chiral product. It may be a diastereomeric product. It may comprise at least about 80% of a single optical isomer, or of a single diastereomer, or at least about 85, 90 or 95% thereof.

### BRIEF DESCRIPTION OF THE DRAWINGS

Preferred embodiments of the present invention will now be described, by way of example only, with reference to the accompanying drawings wherein:

FIG. 1 illustrates the operation of several embodiments of the invention;

FIG. 2 is a scheme illustrating classical kinetic resolution and Dynamic Kinetic Resolution (DKR);

FIG. 3 is a scheme illustrating a part of the glycolytic pathway;

FIG. 4 shows the structure of Zeolite Beta;

FIG. 5 shows the chemical structures of some common polyelectrolytes;

FIG. 6 is a scheme showing Sonogashira cross-coupling catalyzed by Pd nanoreactors;

FIG. 7 shows micrographs of directly-coated zeolite particles;

FIG. 8 is an epifluorescence micrograph of zeolite nanoreactor;

FIG. 9 is a graph showing racemization of (R)-1-phenylethanol for various zeolite catalysts;

FIG. 10 is a scheme illustrating Dynamic Kinetic Resolution of 1-phenylethanol;

FIG. 11 shows micrographs of calcium carbonate templates;

FIG. 12 shows micrographs of hollow polyelectrolyte capsules;

FIG. 13 is an XRD (x-ray diffraction) pattern of Zeolite Beta;

FIG. 14 shows an SEM (scanning electron micrograph) of Zeolite Beta;

FIG. 15 shows micrographs of zeolite-containing templates;

FIG. 16 is a scheme illustrating the mechanism of acid-catalyzed racemization of (R)-1-phenylethanol;

FIG. 17 is a scheme illustrating the racemization and dehydration of (R)-1-phenylethanol;

FIG. 18 is a graph showing selective esterification of 1-phenylethanol against time in different solvents;

FIG. 19 shows the chemical structures of CALB-active substrates and (−)-menthol; and

FIG. 20 is a graph of selective esterification of 1-indanol against time in different solvents.

### DETAILED DESCRIPTION OF PREFERRED EMBODIMENTS

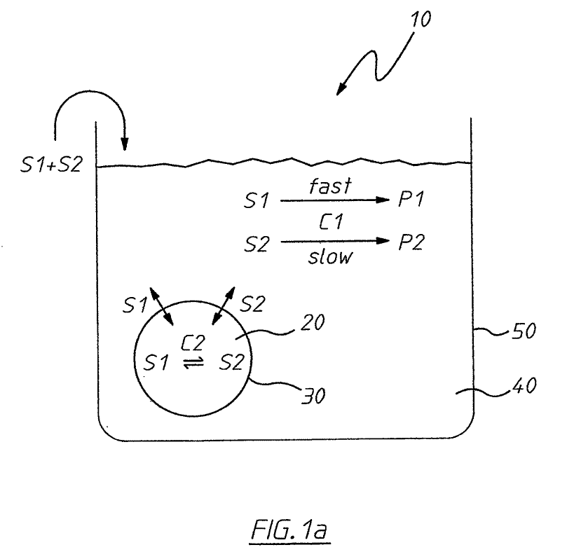

The present invention relates to a reaction system comprising a first reaction promoter and a plurality of microcapsules, each of said microcapsules comprising a second reaction promoter encapsulated within an encapsulant. In the context of the present specification, such terms as “first” reaction promoter, “second” reaction promoter etc. are used to identify different species and should not be interpreted as indicating the order in which these perform their functions in any particular reaction sequence. In the reaction system, the first reaction promoter is capable of converting a first substrate into a first substance and the second reaction promoter is capable of converting a second substrate into a second substance. The second substrate is capable of passing through the encapsulant to contact the second catalyst, and the second substance is then capable of passing out of the microcapsules through the encapsulant. The first substrate and the second product may optionally also be capable of passing through the encapsulant. In some embodiments of the reaction system, either (a) the first substance is the second substrate, or (b) the second substance is the first substrate. In some embodiments, in option (a) conversion (e.g. conversion rate) of the second substance by the first reaction promoter is low, and in option (b) conversion (e.g. conversion rate) of the first substance in the microcapsules are low. In some embodiments the first and second reaction promoters are capable of interacting so as to deactivate one or both thereof, but in the reaction system are at least partially prevented from doing so due to encapsulation of the second reaction promoter. In this context, “at least partially prevented” indicates that the encapsulation prevents or inhibits deactivation of the second reaction promoter. Thus the present invention provides a benefit that when mutually incompatible reaction promoters (e.g. catalysts) are required for a reaction (for example comprising more than one step), those reaction promoters may be prevented from interacting with each other while maintaining the convenience of conducting the reaction in a single reaction vessel. The deactivation of the second reaction promoter by the first reaction promoter, or of the first reaction promoter by the second reaction promoter, in a system according to the present invention relative to the deactivation of the second reaction promoter by the first reaction promoter, or of the first reaction promoter by the second reaction promoter, in a similar system in which the second reaction promoter is not encapsulated and is capable of interacting with the first reaction promoter may, for example, be less than about 50%, or less than about 40, 30, 20, 10, 5, 2, 1, 0.5 or 0.1%, and may be for example about 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45 or 50%, although in some cases it may be greater than about 50%, e.g. between about 50 and about 90%. The deactivation in the reaction system of the present invention may be completely prevented. Examples of embodiments of the invention are illustrated in FIG. 1, described in detail later herein.

In case (a) the rate of conversion of the second substance by the first reaction promoter may be sufficiently low as to allow either separation of the second substance with acceptable yield and/or purity or further reaction of the second substance to a product having acceptable yield and/or purity. Thus a first reaction promoter conversion ratio, defined as the ratio of the rate of conversion of the first substrate by the first reaction promoter to the rate of conversion of the second substrate by the first reaction promoter, may be greater than about 2, or greater than about 3, 4, 5, 6, 7, 8, 9, 10, 15, 20, 50 or 100. It may be between about 2 and 1000, or 5 and 1000, 5 and 1000, 10 and 1000, 50 and 1000, 100 and 1000, 500 and 1000, 2 and 100, 2 and 50, 2 and 20, 2 and 10, 2 and 5, 5 and 100, 10 and 100, 50 and 100 or 10 and 50, e.g. about 2, 3, 4, 5, 6, 7, 8, 9, 10, 15, 20, 25, 30, 35, 40, 45, 50, 60, 70, 80, 90, 100, 200, 300, 400, 500, 600, 700, 800, 900 or 1000, or may be greater than 1000. The acceptable yield, and, independently the acceptable purity, may be greater than about 50%, or greater than about 60, 70, 80, 90, 95 or 99%, for example about 50, 55, 60, 65, 70, 75, 80, 85, 90, 95, 96, 97, 98, 99, 99.5 or 99.9% on a weight or mole basis. In case (a) also, the first reaction promoter may be capable of interconverting the first substrate and the second substrate, i.e. it may be capable of converting the first substrate into the second substrate and of converting the second substrate into the first substrate. Thus for example, the first substrate may be a first optical isomer of a chiral compound, and the second substrate may be the second optical isomer of the chiral compound, and the first reaction promoter may be a catalyst, e.g. an acid catalyst, capable of racemising the chiral compound. If the second reaction promoter is an esterification catalyst, which selectively esterifies the second optical isomer to form an optically active ester, then the first reaction promoter should not racemise the optically active ester to any great extent, to allow separation of a relatively pure product (where relatively pure is defined in terms of the acceptable purity described above).

Similarly, in case (b) the rate of conversion of the first substance in the microcapsules may be sufficiently low as to allow either separation of the first substance with acceptable yield and/or purity or further reaction to a product having acceptable yield and/or purity, where acceptable yield and purity are as described earlier. In this case the conversion of the first substance in the micro capsules (if it occurs) may be into one or more undesirable by-products. In case (b) also, the second reaction promoter may be capable of interconverting the first substrate and the second substrate in the microcapsules, i.e. it may be capable of converting the first substrate into the second substrate and of converting the second substrate into the first substrate. Thus for example, the first substrate may be a first optical isomer of a chiral compound, and the second substrate may be the second optical isomer of the chiral compound, and the second reaction promoter may be a catalyst, e.g. an acid catalyst, capable of racemising the chiral compound, which catalyst is encapsulated in an encapsulant. If the first reaction promoter is an esterification catalyst, which selectively esterifies the first optical isomer to form an optically active ester, then the second reaction promoter may not be capable of racemising the optically active ester to any great extent, to allow separation of a relatively pure product (where relatively pure is defined in terms of the acceptable purity described above).

The reaction system may comprise one or more further reaction promoters (e.g. reagents or catalysts) which may be capable of converting the product of the first two coupled reactions (i.e. the reactions promoted by the first reaction promoter and the microcapsules) into a final product which may subsequently be separated from the reaction system. In this case, the rates of conversion of a final product in the microcapsules and by the first and further reaction promoters may be low, optionally negligible, optionally zero. These rates of conversion may be sufficiently low as to allow separation of the final product in acceptable yield and purity (as defined earlier). These rates may be low, negligible or zero due to either the inability of the second reaction promoter to convert the final product, or due to a physical barrier which at least partially prevents the final product from contacting the second reaction promoter, or both.

The first reaction promoter may be encapsulated or not encapsulated. The first and the second reaction promoters may, independently, comprise one or more facilitating species. If a reaction promoter comprises more than one facilitating species (e.g. 2, 3, 4 or 5 facilitating species) these may operate together so as to promote the reaction promoted by that reaction promoter. Each of said facilitating species may, independently, be a catalyst or a reagent. If a reaction promoter comprises more than one facilitating species, said facilitating species may operate sequentially or cooperatively, or some may act sequentially and some cooperatively. An example of cooperative operation would be if, for example, the first reaction promoter comprised a catalyst and a reagent, whereby the reagent could react with the first substrate under the catalytic influence of the catalyst to form the first substance. An example of sequential operation would be if, for example, the first reaction promoter comprised two catalysts, whereby one catalyst catalysed reaction of the first substrate to an intermediate, and the other catalyst catalysed reaction of the intermediate to the first substance. Thus in sequential operation, a reaction promoter comprising more than one facilitating species would promote a reaction comprising a cascade of individual reaction steps. Other permutations (e.g. two catalysts and a reagent, whereby the reagent could react with the first substrate under the catalytic influence of one of the catalysts to form an intermediate, and the other catalyst catalysed reaction of the intermediate to the first substance) may readily be envisaged by those skilled in the art.

The further reaction promoters (if present) may each, independently, be encapsulated or not encapsulated. An encapsulated reaction promoter may be encapsulated in the same microcapsules as the second reaction promoter (optionally separated from the second reaction promoter, e.g. in different layers of the microcapsules, or in separate loci within the microcapsules), or as any other encapsulated reaction promoter, or in different microcapsules. The encapsulant for any encapsulated reaction promoter may be the same as or different to the encapsulant for any other encapsulated reaction promoter.

The encapsulant(s) should be permeable towards both the substrate for the reaction promoter therein and towards the substance produced by said reaction promoter. The encapsulant may be impermeable, or of low permeability, towards any one or more other components in the reaction system (reaction promoters, reagents, solvents, other substrates, substances and products). In particular, if the first and second reaction promoters are incompatible with each other (i.e. are capable of interacting so as to deactivate one or both thereof) then the encapsulant should be impermeable, or of low permeability, towards the first and second reaction promoters. The encapsulant may incorporate chemical entities which enable selective permeability through the encapsulant. For example, it is known that certain proteins or channel structures are capable of selectively transporting particular species across a membrane, and these proteins or channel structures may be incorporated into the encapsulant for selective permeability therethrough. Each encapsulant, independently, may comprise a polymer or a mixture of polymers, or some other encapsulant. The or each polymer may be a polyelectrolyte. The encapsulant may comprise more than one layer, e.g. 2, 3, 4, 5, 6, 7, 8, 9 or 10 layers. The or each layer may be between about 2 and about 50 nm thick, or between about 2 and 40, 2 and 30, 2 and 20, 5 and 50, 10 and 50, 20 and 50, 5 and 30 or 10 and 30 nm thick, e.g. about 2, 3, 4, 5, 6, 7, 8, 9, 10, 15, 20, 25, 30, 35, 40, 45 or 50 nm thick, or may be greater than 50 nm thick. Each layer may, independently comprise a polymer or a mixture of polymers or some other material. The encapsulant may comprise one or more electrically charged polymeric layers. The encapsulant may comprise one or more positively charged polymeric layers and one or more negatively charged polymeric layers. It may comprise alternating positively and negatively charged polymer layers. In some embodiments the encapsulant, or one or more of the layers thereof, comprise functional groups that encourage penetration of particular substances therethrough. In some embodiments the encapsulant, or one or more of the layers thereof, comprise functional groups that retard or prevent penetration of particular substances therethrough.

The first and second reaction promoters, and, if present, further reaction promoters, may each, independently, be present in the reaction system as a solid, a liquid, a dissolved substance, an emulsified substance, a gas or in any other suitable form. In some embodiments, one of the first and second reaction promoters is a solid and the other is in solution. In particular, the first reaction promoter may be in solution and the second reaction promoter may be a solid. The various substrates, substances and products described herein may each, independently be present in solution.

The present invention also provides a microcapsule comprising a reaction promoter encapsulated within an encapsulant. In particular, the invention provides a microcapsule comprising a reaction promoter encapsulated within an encapsulant, when used in a reaction system according to the present invention, or when used in a method according to the present invention. More particularly there is provided a microcapsule comprising a reaction promoter together with a substrate and product, said reaction promoter, substrate and product being encapsulated within an encapsulant, wherein the reaction promoter is capable of promoting reaction of the substrate to the product. In an example there is provided a microcapsule comprising a zeolite and an enantiomeric pair of benzylic alcohols, said zeolite and pair of alcohols being encapsulated within a polymeric encapsulant, wherein the alcohols are capable of passing through the encapsulant.

The conversion of the first substance (case b above) or the second substance (case a above) by the second reaction promoter may be low either because the rate of conversion of said substance by the second reaction promoter is low, or because said substance passes slowly through the encapsulant to contact the second reaction promoter. The slow rate of passing through the encapsulant may be due to molecular size of the substance, or to polarity, electric charge, hydrophobicity/hydrophilicity, specific affinity or some other property. In cases where one or other of the reaction promoters (or both) are solids, these may be physically prevented from passing through the encapsulant.

The reaction system may additionally comprise a separator for separating a product from the first reaction promoter and from the microcapsules. The separator may comprise a filter, a microfilter, an ultrafilter, an affinity adsorbent, a selectively permeable membrane or some other suitable separator, or may comprise a combination of two or more of these. The nature of suitable separators will be apparent to one skilled in the art from the nature of the product, the first reaction promoter and the microcapsules, optionally together with other properties of the system.

The reaction system may also comprise a purifier for purifying the separated product. The purifier may separate the product from a reagent and/or from a by-product and/or from some other unwanted substance. Again, those skilled in the art will readily appreciate appropriate purifiers. These may for example include distillation apparatus, membrane separation apparatus, chromatographic separators (gc, hplc, gpc, sec, tlc etc.) and others.

The conversion of a substrate to the corresponding substance may be a selective conversion. The conversion of the first substrate to the first substance may be a selective conversion, i.e. the first reaction promoter may be a selective reaction promoter, and may be incapable of promoting conversion of the second substrate, or may promote conversion of the second substrate at a slower rate than of the first substrate. The selectivity of the first reaction promoter (i.e. the rate of conversion of the first substrate by the first reaction promoter divided by the rate of conversion of the second substrate by the first reaction promoter) may be greater than about 1, or greater than about 1.5, 2, 3, 4, 5, 6, 7, 8, 9, 10, 15, 20, 50 or 100. It may be between about 1 and about 100, or between about 2 and 1000, or 5 and 1000, 5 and 1000, 10 and 1000, 50 and 1000, 100 and 1000, 500 and 1000, 2 and 100, 2 and 50, 2 and 20, 2 and 10, 2 and 5, 5 and 100, 10 and 100, 50 and 100 or 10 and 50, e.g. about 1.1, 1.2, 1.3, 1.4, 1.5, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900 or 1000 or may be greater than 1000. The conversion of the second substrate to the second substance may be a selective conversion, i.e. the microcapsules may be incapable of promoting conversion of the first substrate, or may promote conversion of the first substrate at a slower rate than of the second substrate, either because the second reaction promoter is a selective reaction promoter or because the first substrate is incapable of passing through the encapsulant to contact the second reaction promoter, or passes therethrough at a slow rate. The selectivity of the microcapsules (i.e. the rate of conversion of the second substrate in the microcapsules divided by the rate of conversion of the first substrate in the microcapsules) may be greater than about 1, or greater than about 1.5, 2, 3, 4, 5, 6, 7, 8, 9, 10, 15, 20, 50 or 100. It may be between about 1 and about 1000, or between about 2 and 1000, or 5 and 1000, 5 and 1000, 10 and 1000, 50 and 1000, 100 and 1000, 500 and 1000, 2 and 100, 2 and 50, 2 and 20, 2 and 10, 2 and 5, 5 and 100, 10 and 100, 50 and 100 or 10 and 50, e.g. about 1.1, 1.2, 1.3, 1.4, 1.5, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900 or 1000 or may be greater than 1000.

The first and second reaction promoters may be capable of interacting (e.g. reacting) so as to deactivate one or both thereof, but in the reaction system of the present invention may be at least partially prevented from doing so due to encapsulation of the second reaction promoter. For example, if the one reaction promoter is an enzyme and the other reaction promoter is an acid catalyst, the acid catalyst may be capable of deactivating (e.g. denaturing) the enzyme so that it would be incapable of performing its normal function. However encapsulation of the acid catalyst (or alternatively of the enzyme) would at least partially prevent interaction between the two, thereby allowing both to remain active in a single reaction system.

The first reaction promoter may be dispersed, suspended, dissolved or otherwise distributed within a reaction medium, e.g. a solvent. The reaction medium may comprise more than one solvent, or a solvent and a cosolvent. It may comprise a surfactant e.g. an emulsifier. Suitable solvents will depend on one or more of the nature of the first reaction promoter, the substrates, the substances produced by the first reaction promoter and the microcapsules, the microcapsules (particularly the encapsulant) etc. The reaction medium may be organic, either polar or non-polar. It may be aqueous, and may comprise an aqueous solution. It may comprise a combination of organic and aqueous components. It may comprise inorganic non-aqueous components. It may comprise one or more salts as required. The reaction medium may be capable of dissolving, dispersing, suspending or emulsifying the first reaction promoter, and may be capable of dispersing or suspending the microcapsules. The reaction medium may have a suitable polarity to be compatible with the microcapsules.

The microcapsules may have a mean (weight or number average) diameter of between about 0.2 and about 10 microns, or between about 0.2 and 5, 0.2 and 2, 0.2 and 1, 0.5 and 5, 0.5 and 2, 0.5 and 1, 1 and 10, 2 and 10, 5 and 10, 1 and 5 or 2 and 5 microns, e.g. about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 microns, or may be larger than about 10 microns or smaller than about 0.2 microns. If the system comprises more than one type of microcapsule (e.g. if more than one of the reaction promoters are encapsulated in different microcapsules), then each type of microcapsules may, independently, have the mean diameter described above. The microcapsules may be spherical, or approximately spherical, or may be ovoid, polyhedral, oblate spheroid, tablet shaped, rounded, irregular or some other suitable shape. In the event that the microcapsules are not spherical, the diameter described above may be a maximum diameter, a minimum diameter, a mean diameter or some other suitable dimension.

The microcapsules may comprise an energy absorber for absorbing energy (e.g. radiation), optionally for converting the energy to a form in which the energy can be transferred to the second substrate and/or to the second reaction promoter in order to promote the reaction promoted by the second reaction promoter. In some embodiments, the energy absorber is a radiation absorber. It may for example be capable of absorbing radiation so as to raise the temperature locally within the capsules, so as to accelerate the conversion of a substrate by the second reaction promoter. For example the second reaction promoter may comprise a metal which is capable of converting the energy of microwave radiation into heat. The localised temperature within the microcapsules in this case may be between about 40 and 250° C., or between about 40 and 200, 40 and 150, 40 and 100, 40 and 60, 50 and 250, 100 and 250, 150 and 250, 50 and 150, 50 and 100 or 100 and 150° C., e.g. about 40, 50, 60, 70, 80, 90, 100, 120, 140, 160, 180, 200, 220, 240 or 250° C., or some other temperature. In some embodiments, the temperature outside the microcapsules is maintained low, e.g. below about 0, 5, 10, 15, 20 or 25° C., and the localised temperature within the microcapsules is maintained at a higher temperature as to described above. In this case the temperature within the microcapsules may be between about 0 and 40° C., or between 0 and 20, 0 and 10, 10 and 40, 20 and 40, 10 and 30 or 15 and 25° C., e.g. about 0, 5, 10, 15, 20, 25, 30, 35 or 40° C. Alternatively the microcapsules may comprise a photosensitiser or photoinitiator (e.g. a benzoin ether) for absorbing radiation, e.g. UV radiation, in order to catalyse radiation promoted reaction of a substrate. In this case, the encapsulant should be substantially transparent (e.g. at least about 50, 60, 70, 80, 90 or 95% transparent) to the wavelength or wavelengths of radiation absorbed by the photosensitiser or photoinitiator.

In one form, the reaction system of the present invention comprises a first reaction promoter capable of converting a first substrate into a first substance, and a plurality of microcapsules, each of said microcapsules comprising a second reaction promoter encapsulated within an encapsulant. The second reaction promoter is capable of converting a second substrate into a second substance, wherein the second substrate is capable of passing through the encapsulant to contact the second reaction promoter and the second substance is capable of passing out of the microcapsules through the encapsulant. In this particular form, the first and second reaction promoters are capable of interacting so as to deactivate one or both thereof, but in the present reaction system are at least partially prevented from doing so due to encapsulation of the second reaction promoter. Preferably in this form of the invention, the encapsulant is substantially impermeable to the first reaction promoter and to the second reaction promoter. This form of the invention is capable of providing a system in which mutually incompatible reaction promoters can coexist and perform their separate functions without substantial interaction (e.g. deactivation) of one reaction promoter with the other.

In some embodiments, either (a) the first substance is the second substrate, or (b) the second substance is the first substrate. In this case, the system is capable of promoting sequential reactions promoted by the two reaction promoters. The product of these sequential reactions may be further reacted under the influence of a third, and optionally further, reaction promoters. Thus for example a third reaction promoter may be present in the reaction system, encapsulated so as to at least partially prevent interaction with the first reaction promoter. This reaction promoter may be capable of promoting reaction of the product of the second substance (option a above) or the first substance (option b above). Alternatively (or additionally), the reaction system may comprise the third reaction promoter separated from the first reaction promoter and the microcapsules by a selectively permeable membrane capable of at least partially preventing passage of the reaction to promoters and the microcapsules. For example, the DKR system described herein may be isolated from a hydrolysis catalyst by a membrane which at least partially prevents passage of the catalysts and the microcapsules and also at least partially prevents passage of the alcohol optical isomers but permits passage of the ester thereof. In this case, addition of racemic alcohol to the DKR side of the membrane would produce the ester of a single optical isomer of the alcohol as described elsewhere herein. This ester could then pass through the membrane where it would be hydrolysed by the hydrolysis catalyst to regenerate only a single optical isomer of the alcohol. If in this example the hydrolysis catalyst were microencapsulated, this would facilitate isolation of the single optical isomer of the alcohol which was the desired product. This is illustrated in FIG. 1b, described in detail later herein.

In another embodiment of this form of the invention, a substance produced by one of the reaction promoters could pass through a selective membrane to a third reaction promoter, and the substance produced thereby could pass back through the membrane to act as the substrate for the other reaction promoter. This is illustrated in FIG. 1c, described in detail later herein.

The present invention also provides a method for conducting a reaction using a reaction system as described above. When either the first substrate or the second substrate or both the first substrate and the second substrate is added to the reaction system, either (a) the first substrate is converted to the second substrate and the second substrate is converted to the second substance, or (b) the second substrate is converted to the first substrate and the first substrate is converted to the first substance.

The reaction system may be stirred, shaken, mixed, sonicated or otherwise agitated in order to facilitate efficient contact between relevant components of the system and substrates. The agitation should not be sufficiently vigorous as to cause the micro capsules to rupture. The reactor of the invention may therefore comprise an agitator, e.g. a stirrer, shaker, mixer, sonicator or other agitator. The method may be conducted at any suitable temperature and pressure that does not cause substantial damage to components of the reaction system. Commonly atmospheric pressure will be used, but those skilled in the art will readily appreciate when a different pressure (e.g. elevated pressure) is required for reaction. If elevated pressure is required, the reactor of the present invention may comprise a pressure vessel for containing the reaction system. The temperature of the reaction system should be sufficient to achieve the required conversion in an acceptable time, without causing deactivation and/or degradation (e.g. denaturation, conversion to to unwanted by-products etc.) of the reaction promoters, the encapsulant, the substrates, the substances produced by the reaction promoters and the final product (if separate from the substances produced by the reaction promoters). In this context, deactivation refers to conversion of a species to a form in which it is incapable, or less capable, of performing its normal function. The reduction in performance may, for example, be at least about 50, 60, 70, 80, 90 or 95%, and may be about 50, 55, 60, 65, 70, 75, 80, 85, 90, 91, 92, 93, 94, 95, 96, 97, 98, 99, 99.5 or 99.9%, although in some cases it may be less than about 50%, e.g. between about 10 and about 50%. Deactivation of a reaction promoter may therefore refer to inhibition thereof. Deactivation may refer in this context to complete deactivation, i.e. the case in which the reduction in performance is 100%. The temperature is commonly between about 0 and 100° C., or between about 0 and 50, 0 and 20, 0 and 10, 10 and 100, 20 and 100, 50 and 100, 10 and 90, 10 and 50, 20 and 50 or 20 and 40° C., e.g. about 0, 5, 10, 15, 20, 25, 30, 35, 40, 45, 50, 60, 70, 80, 90 or 100° C., although on occasions the temperature may be above 100° C. or below 0° C. The temperature may be room temperature or ambient temperature. The reactor may therefore incorporate a temperature controller, for controlling the temperature of the reaction system. The temperature controller may comprise a heater and/or a cooler and may comprise a control unit.

The method may comprise irradiating the reaction system with radiation of a wavelength capable of being absorbed by a component of the reaction system, by the first or second substrate or by more than one of these. The irradiation may be with IR, UV, visible, microwave, ultrasound or other radiation as appropriate.

An embodiment of the invention is illustrated in FIG. 1a. In FIG. 1a, system 10 comprises first catalyst C1 capable of converting a first substrate S1 to a product P1 at a rate greater than it converts a second substrate S2 to a byproduct P2. System 10 also comprises a plurality of microcapsules 20, only one of which is shown in FIG. 1a for purposes of simplicity. Each microcapsule 20 comprises second catalyst C2 encapsulated within an encapsulant 30. Catalyst C2 is capable of interconverting substrates S1 and S2. Substrates S1 and S2 are capable of passing through encapsulant 30. Product P1 is incapable of being converted by catalyst C2 to byproduct P2, optionally because P2 can not pass through encapsulant 30. Catalyst C1 is suspended or dissolved in a carrier liquid 40, and microcapsules 20 are suspended in carrier liquid 40. The suspending may be assisted by a stirrer, not shown in FIG. 1a. Substrates S1 and S2 and product P1 may also be soluble in liquid 40. Reaction system 10 may be contained in container 50.

In operation, a mixture of S1 and S2 (commonly an equimolar mixture) is added to to reaction system 10. S1 is converted to product P1, whereas conversion of S2 to byproduct P2 is considerably slower. P1 is not converted by either catalyst C1 or C2, and consequently accumulates in system 10. This results in an excess of S2 over S1 in system 10, due to depletion of S1 by conversion to P1. S1 and S2 can pass through encapsulant 30 to contact catalyst C2, which interconverts S1 and S2 to regenerate an equimolar mixture of S1 and S2. This mixture can then pass out of microcapsules 20. This process generates a continuous supply of substrate S1 from S2 to be converted to product P1. In the absence of interconverting catalyst C2, a maximum yield of product P1 from an equimolar mixture of S1 and S2 would be 50%, however, since C2 provides a supply of additional S1 from S2, yields of greater than 50% are possible.

Another embodiment is described in FIG. 1b. In FIG. 1b, system 100 comprises DKR side 110 and hydrolysis side 120, separated by selective membrane 130 and contained in vessel 140. Side 110 contains esterification catalyst C1, which is capable of selectively catalysing reaction of alcohol isomer A1 with reagent R to form optically active ester E1. Side 110 also contains equilibration catalyst C2, encapsulated within encapsulant 150. Catalysts C1 and C2 are capable of interacting so as to at least partially deactivate C1, however are at least partially prevented from doing so by the encapsulation of C2. Encapsulant 150 prevents passage of catalysts C1 and C2, and preferably also of ester E1, but permits passage of alcohols A1 and A2. Side 120 comprises hydrolysis catalyst C3, which is capable of hydrolysing ester E1 to regenerate alcohol optical isomer A1. Selective membrane 130 separates side 110 from side 120, and is capable of at least partially preventing passage of catalyst C1, as well as of alcohol optical isomers A1 and A2 and also of the microcapsules which comprise catalyst C2. Membrane 130 is capable of permitting passage of ester E1.

In operation of system 100, when a mixture of optical isomers A1 and A2, or optionally only isomer A2, is added to side 110 of vessel 140, A1 (if present) and A2 enter the microcapsules through encapsulant 150 and are equilibrated by catalyst C2. A1 and A2 pass out of the microcapsules, where catalyst C1 catalyses esterification of A1 to E1. A2 is reequilibrated with A1 by penetrating the encapsulant and equilibrating with A1 under the influence of C2. E1 passes through membrane 130 to side 120, and is hydrolysed by catalyst C3 to regenerate A1. A1 is incapable of passing through membrane 130, and is therefore trapped in side 120 of system 100. The system therefore provides means to selectively generate A1 from either A2 or from a mixture of A1 and A2. As A1 is continually removed from DKR side 110 by conversion to E1, A2 is continually converted to A1 by catalyst C2.

A further embodiment is illustrated in FIG. 1c. In FIG. 1c, reaction system 200 comprises first catalyst C1 capable of converting a first substrate S1 into a first substance P1. System 200 also comprises a plurality of microcapsules 210 (only one of which is shown for reasons of simplicity). Each microcapsule 210 comprises second catalyst C2 encapsulated within encapsulant 220, said second reaction promoter being capable of converting second substrate S2 into second substance P2. Second substrate S2 is capable of passing through encapsulant 220 to contact second catalyst C2 and second substance P2 is capable of passing out of microcapsules 210 through encapsulant 220. In this example, first and second catalysts C1 and C2 are capable of interacting so as to deactivate one or both thereof, but in reaction system 200 are at least partially prevented from doing so due to encapsulation of second reaction promoter C2 by encapsulant 220. Encapsulant 220 is impermeable to first reaction promoter C1 and to second reaction promoter C2. Catalysts C1 and C2 are located in carrier liquid 230 within vessel 240. Vessel 240 also comprises membrane 250, and catalyst C3 (optionally also located in carrier liquid 230 or some other carrier liquid). Membrane 250 separates catalyst C1 and microcapsules 210 from catalyst C3, as it is impermeable to them. This separation may be for the purpose of preventing interaction between catalysts C1 and C2 with catalyst C3, or may be for ease of separation of product P2, or may be for some other purpose, for example prevention of a byproduct of conversion of P1 to S2 from contacting either C1 or C2. Membrane 250 is capable of allowing passage of substance P1 and of substrate S2. It may optionally be impermeable to one or more of S1, P2 and a byproduct of the conversion of P1 to S2. Catalyst C3 is capable of catalysing conversion of substance P1 to substrate S2.

In operation, when substrate S1 is added to vessel 240, it is converted by C1 to P1. P1 then passes through membrane 250 and is converted by C3 to S2. S2 then passes through membrane 250 and into microcapsules 210 (through encapsulant 220), where it is converted by C2 to P2. In this example P2 is the final product of the reaction. In some cases, P2 may be sensitive to catalyst C3, and may be at least partially prevented from contacting C3 by membrane 250 through which it may be incapable of passing.

Yet another embodiment of the invention is illustrated in FIG. 1d. In FIG. 1d, system 300 comprises reaction promoters C1, C2, C3 and C4, capable of promoting reaction of substrate S1 to product P1, S2 to product P2, S3 to product P4 and S4 to product P4 respectively. Microcapsules M2 and M4 comprise reaction promoters C2 and C4 respectively, encapsulated within encapsulants E2 and E4 respectively. For the purpose of simplicity, only one of each microcapsule M2 and M4 is shown, however in practice a plurality of each are present. System 300 is located within vessel 310, which comprises two chambers 315 and 320, separated by selectively permeable membrane 325. Catalyst C1 and microcapsules M2 are located in chamber 315 and catalyst C3 and microcapsules M4 are located in chamber 320. Chambers 315 and 320 also comprise a carrier liquid, optionally a solvent, and catalysts C1 and C3, and microcapsules M2 and M4 are dispersed in the carrier liquid. Commonly in system 300, C1 and C2 are incompatible (i.e. may interact to deactivate one or other thereof) and C3 and C4 are incompatible. Alternatively, microencapsulation of C2 and C4 may be for the purpose of simplifying product separation. In system 300, S2 and S4 are capable of passing through encapsulants E2 and E4 respectively in order to enter microcapsules M2 and M4 respectively, and P2 and P4 are capable of passing through encapsulants E2 and E4 respectively in order to exit microcapsules M2 and M4 respectively. Membrane 325 is commonly impermeable to catalysts C1 and C3 and microcapsules M2 and M4, although in some modes this is not the case.

It will be readily apparent that there are multiple modes in which system 300 may be operated. For example in one mode, product P1 acts as substrate S2, product P2 acts as substrate S3 and product P3 acts as substrate S4. In this mode, addition of substrate S1 to chamber 315 results in conversion of S1 to S2 by C1. S2 then enters microcapsule M2 through encapsulant E2 and is converted by reaction promoter C2 to S3, which then exits M2 through E2 and passes through membrane 325. It is then converted by C3 to form S4. Entry of S4 into microcapsules M4 through encapsulant E4 leads to conversion by C4 into P4, which then exits M4 via E4 as the final product of the cascade of reactions. This mode may be useful, for example if C3 and C4 are incompatible and C1 and C2 are incompatible, and if P4 is not compatible with C1 and can not penetrate membrane 325. Alternatively, this mode may be useful in simplifying separation of the final product P4 from S1 and S2, and/or from C1. In another possible mode, C2 is an equilibration catalyst for S1 and S2 and C4 is an equilibration catalyst for S3 and S4. In this mode, P2 acts as S1, P1 acts as S4 and P4 acts as S3. Thus in operation, when S1 and S2 are added together to chamber 315, S1 is converted to P1 by C1, and S2 enters microcapsules M2 where it is equilibrated with S1 to provide additional feed material for catalyst C1 to produce P1. P1 then passes through membrane 325 into chamber 320. It then enters microcapsules M4, where it acts as S4, to be equilibrated with S3 (i.e. P4). S3 exits microcapsules M4 and is converted by catalyst C3 into final product P3. As noted above, many other modes of operation of system 300 may readily be envisaged. Additionally, further chambers may be added, separated from previous chambers by selectively permeable membranes and containing encapsulated and/or unencapsulated reaction promoters, in order to conduct more complex cascades of reaction.

In an embodiment of the present invention, an encapsulated catalyst causes racemisation of an optical centre in a chiral molecule, and another catalyst provides selective reaction of one of the optical isomers. It will be appreciated that many compounds comprise two or more centres which may be isomerised, either by the same mechanism or by different mechanisms. For example a molecule may comprise two asymmetric centres, providing the possibility of pairs of diastereomers, or it may comprise an asymmetric centre and a double bond, providing the possibility of optical isomers of both cis and trans isomers of the double bond. In the latter case, for example, this embodiment may be extended, so that two equilibration catalysts exist, one for equilibrating each of the sites of isomerism (i.e. the double bond or the asymmetric centre), and two selective conversion catalysts, one each for selectively converting a specific configuration of the sites of isomerism. In this way, this embodiment may provide a means to selectively provide one or more products from a mixture of diastereomers or from other compounds having two sites of isomerism. Clearly, other related modes of operation may be envisaged.

In some embodiments the present invention relates to a novel system allowing the coexistence of otherwise incompatible catalysts, namely the use of polymer nanocapsules to surround and protect one catalyst from another.

Polymer nano capsules and their fabrication constitute an exciting area of research with applications in drug/DNA delivery, biosensorics, polymer chemistry, nanoparticle synthesis, and catalysis. In particular, the fabrication of polyelectrolyte (PE) nanocapsules using Layer-by-layer (LbL) self-assembly onto an appropriate template has been extensively studied. The resulting polyelectrolyte multilayer capsule membranes have been found to be robust, stable in a variety of solvents, and selectively permeable to small molecules but not macromolecules. These properties make hollow polyelectrolyte nanocapsules excellent candidates for catalytic nanoreactors, as they can encapsulate macromolecular catalysts (such as enzymes or nanoparticles) while allowing small molecular substrates and products to traverse the membrane readily.

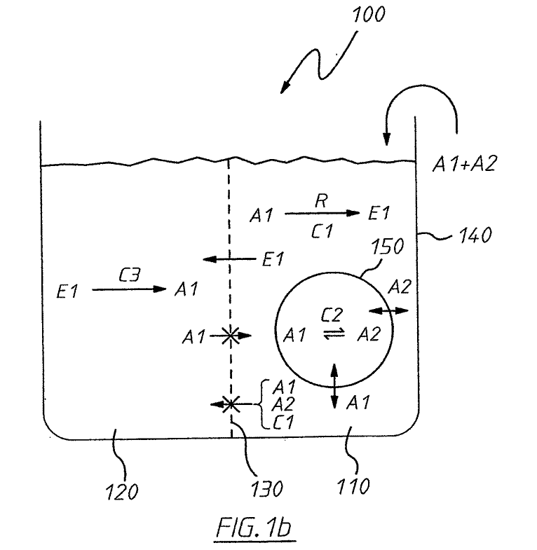

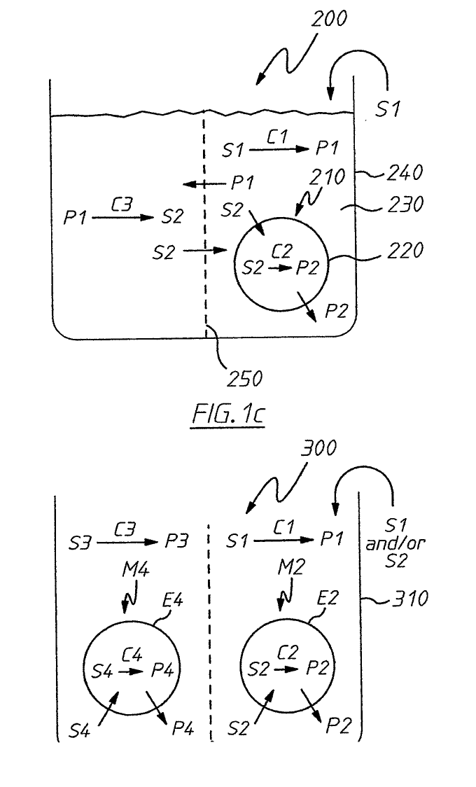

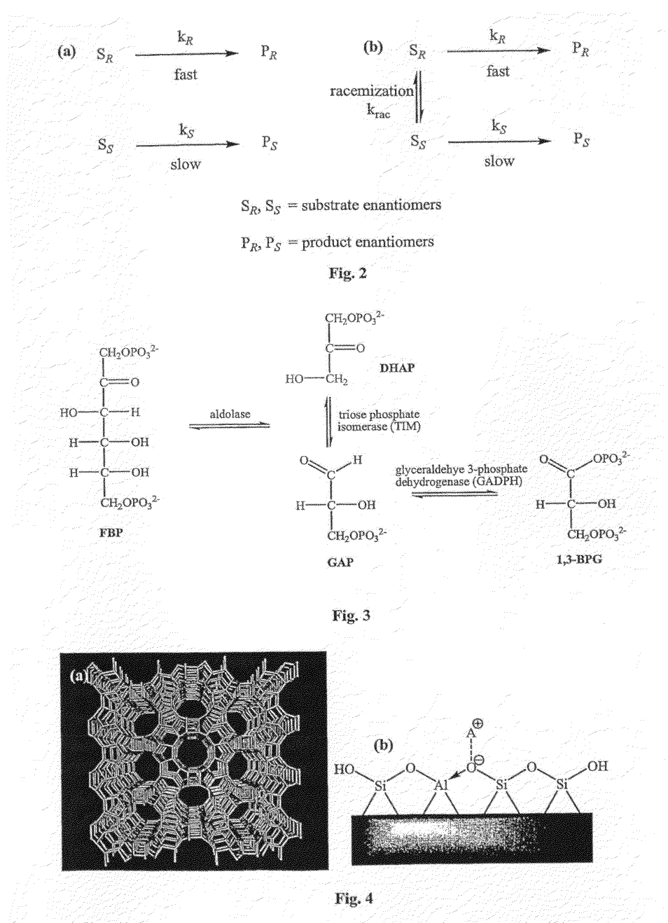

One means of obtaining pure enantiomers is kinetic resolution of the chiral pool. This entails a selective catalyst that converts one enantiomer of a racemic mixture to its product much faster than it does the other enantiomer (FIG. 2(a)). FIG. 2 shows (a) classical kinetic resolution, in which substrate S_{R} is converted to product P_{R} faster than substrate S_{S} is converted to product P_{S} (by an enzyme, for example), generating an enantiomeric excess of product P_{R}, and (b) Dynamic Kinetic Resolution, in which the resolution step occurs as in (a), generating an enantiomeric excess of substrate S_{S} and product P_{R}. A racemization catalyst, however, simultaneously converts the excess S_{S} enantiomer to S_{R} in situ. If the resolution step is very selective (k_{R}>>k_{S}) and much slower than the racemization reaction (k_{rac}>>k_{R}, k_{S}), a near quantitative yield of P_{R} can be obtained.

Classical kinetic resolution is limited, however, to a maximum theoretical yield of 50%. Dynamic Kinetic Resolution (DKR) improves upon this method by incorporation of in situ racemization of the starting material in order to give, theoretically, a quantitative yield of one enantiomer (FIG. 2(b)). Naturally-occurring processes involving the combination of isomerization catalysts and selective resolution catalysts are known (FIG. 3). FIG. 3 shows part of the glycolytic pathway, in which fructose-1,6-bisphosphate (FBP) is cleaved by the enzyme aldolase to form the isomeric products GAP and DHAP. These products are isomerized by the enzyme TIM and only the isomer GAP is simultaneously converted to 1,3-bisphosphoglycerate (1,3-BPG) by the enzyme GADPH.

DKR of secondary alcohols in particular has been studied extensively. These reactions have been conducted generally by using a lipase as a selective esterification catalyst to perform the resolution step, in combination with a racemization catalyst. The racemization catalysts have included rhodium, palladium and ruthenium species. In fact, the combination of the CALB enzyme with a ruthenium racemization catalyst has proven a very effective and general method of DKR, with application to a variety of secondary-alcohol substrates, as well as diols and chiral primary amines. DKR of benzylic secondary alcohols using CALB with the solid acid Zeolite H-Beta as the racemization catalyst has also been reported, but involved sequestering the zeolite from the pH-sensitive enzyme via a biphasic system. This illustrates the crucial importance of mutual tolerance between the catalysts in a DKR reaction, and even the highly successful class of ruthenium racemization catalysts has been investigated thoroughly to devise catalysts more compatible with the enzyme.

Candida antarctica Lipase B (CALB) is a highly versatile enzymatic catalyst used in to a wide variety of chemical syntheses. It can be isolated from the yeast Candida antarctica and performs the highly selective hydrolysis of triglycerides. CALB is composed of 317 amino acid residues and has a molecular weight of 33 kDa. It is a globular protein with approximate dimensions of 30 Å×40 Å×50 Å and contains a very restricted entrance to the active site which is believed to be responsible for the high substrate selectivity and stereoselectivity of the enzyme. CALB also catalyzes the selective esterification (and hydrolysis) of a wide variety of substrates, and has displayed activity in many different organic media, making it a very attractive selective catalyst for use in organic synthesis.

Zeolite Beta is a porous aluminosilicate material that possesses a 3D, negatively charged framework offset by a large number of cations necessary to maintain charge balance (see FIG. 4). Its pores constitute channels of diameter 6.6 or 5.6 Å that propagate perpendicular to one another throughout the structure. First synthesized in 1967, the material has been used widely for various catalytic reactions of hydrocarbons, such as cracking, alkylation, isomerization and disproportionation. Zeolite Beta is easily prepared and the synthetic procedure can be varied to give nanocrystals as small as 200 nm. When charge balance is achieved using protons as counterions the material becomes highly acidic. This acid form, Zeolite H-Beta, exhibits strong, localized acidity that does not leak into an aqueous solvent. FIG. 4 shows the structure of Zeolite Beta, showing (a) a framework structure showing pore channels: Si/Al atoms at vertices, O atoms at midpoints of lines; and (b) a schematic representation of the structure showing Al dopant in the silica network giving rise to a negative charge on the framework, and thus counterions (A⁺).

The localized acidity of Zeolite H-Beta makes it an ideal acid catalyst for encapsulation, ensuring that any acid-catalyzed reaction will occur only within the capsule. Moreover, as a heterogeneous catalyst, the zeolite can be retained easily within a capsule that is still porous to small-molecule substrates and products. Zeolites are also advantageous catalysts for encapsulation in their capacity as bifunctional catalysts, which combine both acid and hydrogenation-dehydrogenation properties. Often, an acidic zeolite is loaded with finely dispersed metal, e.g. platinum or palladium metal, yielding a material that possesses both of these catalytic activities. These bifunctional catalysts have been widely used for various catalytic reactions of hydrocarbons. The ability of the single zeolite to support dual catalytic functions combined with the use of selective membranes in encapsulation could give rise to a novel system for selective catalysis. For example, a ketone could be reduced to a secondary alcohol and this product racemized in situ by the dual hydrogenation-dehydrogenation activity and acidity of an encapsulated bifunctional to catalyst. In addition, the catalyst could be encapsulated with a selective membrane that allowed only one product enantiomer to escape. Such a system could provide selective reduction, for instance, with theoretical yields up to 100% without the need for expensive and synthetically challenging traditional selective catalysts.

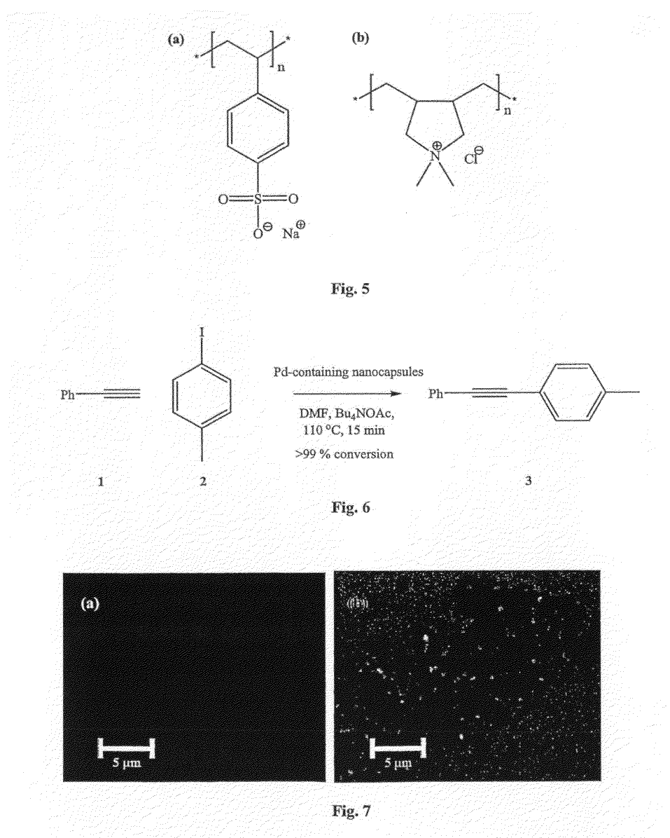

Layer-by-Layer (LbL) assembly entails the alternate adsorption of positively and negatively charged species onto a surface to form a robust, multilayer film. Polyelectrolytes (PE), polymers with monomeric units that contain an electrolyte group, are well suited to this task. They exhibit good adhesion to most surfaces and self-regulation of film thickness caused by electrostatic repulsion between like-charged species in solution during the coating process. Some examples of polyelectrolytes commonly used in the literature include poly(acrylic acid) (PAA), poly(allylamine hydrochloride) (PAH), poly(diallyldimethylammonium chloride) (PDA) and poly(sodium 4-styrenesulfonate) (PSS) (FIG. 5). FIG. 5 shows the structures of polyelectrolytes (a) poly(sodium 4-styrenesulfonate) (PSS) and (b) poly(diallyldimethylammonium chloride) (PDA).

LbL assembly was originally used to fabricate thin, polyelectrolyte films on flat surfaces and has been recently adapted to generate multilayer films on colloidal particles. By using an organic or salt colloidal template, it is possible to produce nanocapsules, which can be rendered hollow via dissolution of the template. The resulting nanocapsules are stable in solution and tend to retain the shape of their template. While stretching and drying can deform the capsules, these effects are generally reversed by addition of water.

The multilayer PE capsule membranes have also demonstrated the very useful property of semipermeability, excluding high molecular weight molecules but allowing diffusion of low molecular weight species. This property is important in allowing encapsulation of macromolecular species such as enzymes and heterogeneous catalysts.

Polyelectrolyte nanocapsules have a host of applications in drug/DNA delivery, biosensorics, polymer chemistry, nanoparticle synthesis, and catalysis. LbL assembly has allowed encapsulation of catalysts to form cell-like nanoreactors that have a number of useful properties. Given the semipermeability of PE capsules, careful selection of the membrane constituents can yield a catalytic system that protects the catalyst from the environment external to the capsule while allowing the desired substrates to diffuse into the capsule and access the catalyst. Capsules have commonly been used heretofore in isolation as templates or chambers for the assembly of nanoparticles or polymeric structures. Capsules with catalytic materials embedded in their membranes have been used successfully as nanoreactors, as shown in FIG. 6. FIG. 6 illustrates Sonogashira cross-coupling reaction between phenylacetylene (1) and 4-iodotoluene (2) catalyzed by nanocapsules containing Pd clusters (2 mol %) in their membranes. The product, p-methyldiphenylacetylene (3), was formed with >99% selectivity. Moreover, there has been considerable interest in the encapsulation of enzymes for use in biosynthesis, and catalytically active enzyme-containing nanoreactors have been successfully prepared. Furthermore, these nanoreactors have been found to protect the encapsulated enzyme from high molecular weight inhibitors, which cannot traverse the semipermeable capsule membrane. Moreover, the use of a chiral, selectively permeable membrane surrounding a catalyst could generate an enantiomeric excess in stereocentre-generating reactions. In the past, thin, optically active polyelectrolyte membranes have demonstrated some enantioselectivity and, therefore, could be excellent candidates for such membranes. Ultimately, the use of chiral components in the membrane could give rise to enantioselection in the reaction via selective permeation of the reagent and/or product enantiomers, thus providing a novel route to asymmetric catalysis.

The impermeability of polyelectrolyte membranes to macromolecular species gives rise to applications in catalyst compartmentalization via encapsulation. The ability to combine multiple, mutually-interfering catalysts within a single reaction system has long been the subject of much investigation, particularly in the area of Dynamic Kinetic Resolution. Past solutions to the problem have included membrane reactors, biphasic systems, and sequential addition of the catalysts. However, the present invention discloses that by reducing the complete assembly of a membrane reactor to truly nanoscopic dimensions, a generic, one-pot, monophasic solution (with its associated benefits) can be found.

Herein is described the use of polyelectrolyte capsules to protect the pH-sensitive enzyme Candida antarctica lipase B (CALB) from the solid acid Zeolite H-Beta by encapsulation of the latter, thereby enabling the use of both catalysts in a one-pot Dynamic Kinetic Resolution (DKR) of secondary alcohols. Using this method, yields were obtained that unambiguously crossed the 50% maximum theoretical yield of classical kinetic resolution (thus proving the operation of Dynamic Kinetic Resolution). Furthermore, the protective capability of the capsules was demonstrated.

The polyelectrolytes poly(diallyldimethylammonium chloride) (PDA) and poly(sodium 4-styrenesulfonate) (PSS) were alternately deposited on the surface of Zeolite H-Beta particles using the LbL method described in the literature. Encapsulation of the zeolite particles was confirmed using fluorescence microscopy, since the capsule membrane exhibited a weak blue fluorescence when excited in the UV range (FIG. 7). FIG. 7 shows (×100 magnification) of directly-coated zeolite particles.

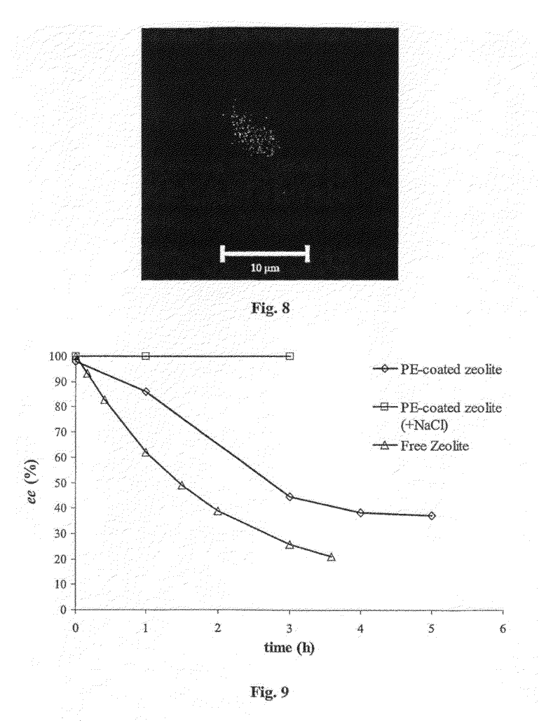

As an alternate route to capsular nanoreactors, calcium carbonate was precipitated onto zeolite nanoparticles, yielding spherical templates with average dimensions of 8-10 microns. Upon PE-coating of these templates and dissolution of the calcium carbonate matrix, hollow capsules containing “loose” zeolite particles were obtained. Fluorescence tagging of the zeolite particles by electrostatic immobilization of the fluorophore [Ru(bipy)₃]²⁺ via ion-exchange prior to encapsulation allowed for examination of the resulting nanoreactors using fluorescence microscopy (FIG. 8). FIG. 8 shows an epifluorescence micrograph (×100 magnification) of a zeolite nanoreactor showing fluorescence-tagged zeolite surrounded by a polyelectrolyte capsule.

These nanoreactors were tested as catalysts for the racemization of secondary alcohols, a reaction that acid zeolites (Zeolite H-Beta in particular) are known to catalyze well. The nanoreactors prepared via calcium carbonate templates did not show any activity, due to quenching of the acidic zeolites by the basic templates. FIG. 9 shows the progress over time of racemization of (R)-1-phenylethanol for various zeolite catalysts. As shown in FIG. 9, the directly coated zeolite retained its catalytic activity with only minor diminution due to substrate diffusion through the PE capsule membrane. Reaction conditions were (R)-1-phenylethanol (100 μL, 0.827 mmol), zeolite catalyst (10 mg), n-dodecane (internal standard, 100 μL, 0.439 mmol), toluene (50 mL), air atmosphere, 60° C.

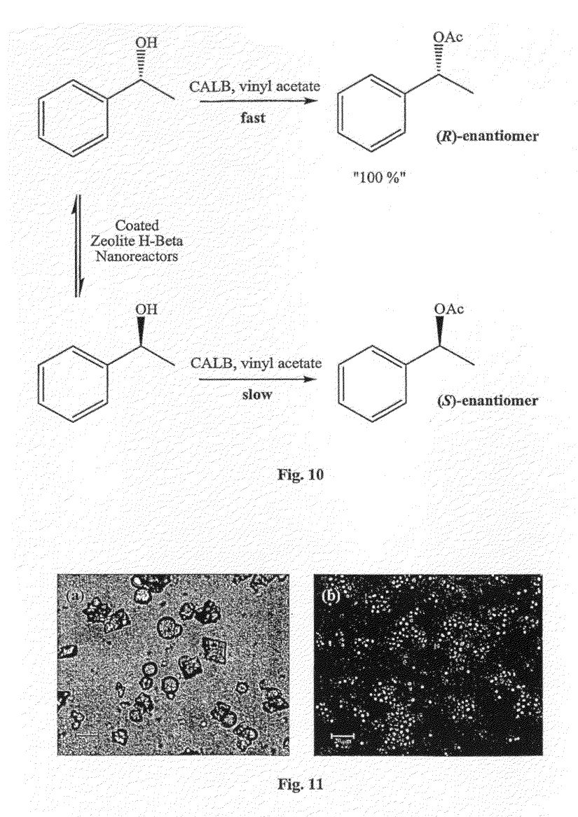

Having obtained catalytically active zeolite nanoreactors for the racemization, it remained to combine them with the enzyme CALB to perform the DKR of secondary alcohols (see FIG. 10). FIG. 10 shows a scheme illustrating Dynamic Kinetic Resolution of 1-phenylethanol, using Zeolite H-Beta nanoreactors and CALB, with vinyl acetate as the acyl donor.

This zeolite/enzyme combination of catalysts has been used previously for this reaction, but a biphasic system and enzyme immobilization were required to achieve macroscopic separation of the catalysts from each other. As for most enzymes, the activity and selectivity of CALB are very sensitive to pH, and the enzyme was therefore expected to be incompatible with the acidic zeolite. It was intended that by using zeolite nanoreactors with membranes impermeable to the macromolecular enzyme both catalysts could be incorporated in a single system to perform DKR.

The DKR reaction was attempted using a variety of solvents, substrates and substrate/catalyst concentration ratios. For each set of reaction conditions, two controls were employed: one with unencapsulated zeolite instead of zeolite nanoreactors and the other with no zeolite. These controls were used to assess the effect of the nanocapsule on catalyst compatibility.

The results gave several trends. First, yields and ee's of the enzyme-selected ester were generally worse when free, unencapsulated zeolite was added to the enzyme, suggesting the acidic zeolite was indeed interfering with the pH-sensitive enzyme. Secondly, the capsules, when employed, significantly attenuated this interference, giving markedly higher yields and ee's for the reactions using nanoreactors rather than unencapsulated zeolite. It appeared that this was due to the exclusion of the macromolecular enzyme from the nanoreactors' interior and, thus, to its protection from the acidic zeolite. Separate tests showed no significant racemization of the ester product over time (as expected), nor did the zeolite catalyze significant achiral esterification on its own, thereby confirming the attribution of reduction in ee (enantiomeric excess) and yield to the impairment of the enzyme. The PE capsules themselves did not exhibit any catalytic activity.

Thirdly, by tuning the relative concentrations of zeolite nanoreactors and CALB, yields of the desired product enantiomer were obtained that unambiguously crossed the 50% bather of classical resolution, thus proving the occurrence of DKR. For the two substrates tested, the best results obtained were a 63% yield of the (R)-ester (70% overall yield of the ester) in 81% ee for 1-phenylethanol and a 57% yield of the selected ester enantiomer (59% overall yield of the ester) in 92 ee for 1-indanol. Although roughly quantitative conversions were obtained much starting material was lost to the dehydration side-reaction, limiting the overall ester yield. Further optimization of the reaction (in particular suppression of the competing dehydration reaction, e.g. through the use of water-rich solvents) may lead to even better yields and enantioselectivities.

In summary, the inventors have prepared catalytically active nanoreactors and used them to enable a new catalytic system for the DKR of secondary alcohols. More generally, this represents a novel means of catalyst protection that may allow the more extensive and flexible use of multi-catalytic systems in the future. Zeolite H-Beta particles were encapsulated using LbL deposition of polyelectrolytes and the resulting nanoreactors were found to be catalytically active. Nanoreactors with larger interior volumes were also prepared using a calcium carbonate template (which was dissolved after coating) surrounding the zeolite.

Furthermore, the nanoreactors were combined with the enzyme CALB to provide a new route towards the Dynamic Kinetic Resolution of secondary alcohols. The reaction was conducted successfully for 1-phenylethanol and 1-indanol. Yields of product up to 70% have been achieved. Enantiomeric excess of up to 92% has been achieved.

Furthermore, the encapsulation of the catalyst improved the product yield and ee significantly, suggesting that the capsule membrane actively protects the pH-sensitive enzyme from the acidic zeolite. This demonstrates the utility of polyelectrolyte nanoreactors not only in DKR but also for catalyst protection and reaction compartmentalization more generally.

The ability to incorporate mutually-interfering catalysts in a single system and to compartmentalize reactions has broad applications, particularly to cascade reactions and DKR.

### EXAMPLES

The examples illustrate the application of the invention to Dynamic Kinetic Resolution. The synthesis of the capsules is described. Zeolite H-Beta and Candida Antarctica lipase B (CALB) are presented as two mutually-interfering catalysts (due to the acidity of the former and the pH-sensitivity of the latter) that could perform Dynamic Kinetic Resolution of secondary alcohols if combined in a single system. Zeolite H-Beta, as a solid acid catalyst that displays very localized acidity even in aqueous systems, is targeted for encapsulation, and the various methods of forming the corresponding polyelectrolyte nanoreactors are discussed. These nanoreactors are characterized and finally combined with the lipase for the successful Dynamic Kinetic Resolution of several secondary alcohols, thereby demonstrating a new means of catalyst protection and separation in multi-catalytic systems.

One of the most common synthetic strategies for the preparation of nanocapsules is the coating of a template with alternating layers of positively and negatively charged polyelectrolytes, followed by dissolution of the template. Calcium carbonate is a particularly convenient template because small (1-10 μm), spherical particles are easily prepared and can eventually be removed simply by dissolution in EDTA solution. This mild removal of the template contrasts with the harsher acidic conditions required by some colloidal templates.

Calcium carbonate core templates were obtained by precipitation after combining aqueous solutions of calcium chloride and sodium carbonate with rapid stirring, according to literature methods. FIG. 11 shows transmitted light micrographs (×40 magnification) of calcium carbonate templates of different morphologies: (a) a mixture of spheres and blocks (particle size 5-20 μm). (b) a sample predominantly of spheres (average diameter ˜5 μm). Thus, upon examination with light microscopy, the particle sizes and morphologies of the templates were found to vary quite significantly with the rate and time of stirring, giving amorphous spheres, crystalline blocks, or, most commonly, some mixture of the two (see FIG. 11(a)). Particle sizes varied between 5-30 μm. By slow addition of the calcium chloride solution to the sodium carbonate solution over 3 min with stirring at 500 rpm, templates with more uniform spherical morphology and average diameter 5 μm (determined by light microscopy, shown in FIG. 11(b)) were obtained.

Hollow polyelectrolyte nanocapsules were prepared by LbL coating of calcium carbonate core templates with several alternate treatments of poly(diallyldimethylammonium chloride) (PDA) and poly(sodium 4-styrenesulfonate) (PSS) solutions, followed by dissolution of the core with EDTA solution. FIG. 12 shows micrographs of hollow polyelectrolyte capsules: (a) transmitted light micrograph; (b) epifluorescence micrograph of the same field; and (c) SEM of capsules, collapsed and aggregated under the vacuum conditions required for imaging. The capsules were examined using transmitted light microscopy and found to retain the spherical morphology of the template and to have a diameter of about 2-3 μm, as shown in FIG. 12(a). The capsules were also examined using epifluorescence microscopy and exhibited a weak blue fluorescence when excited in the UV range, which can be attributed to the fluorescent PSS polyelectrolyte (FIG. 12(b)). SEM images of the capsules were obtained, showing some distortion of the morphology of the capsules upon drying (FIG. 12(c)). While these capsules were composed of 3 PDA/PSS bilayers, the fabrication process involved the deposition of one layer at a time and, therefore, allowed precise control over the number of PE layers and hence the capsule thickness. This method also gave control over the interior, exterior, and net charge on the nanocapsules, which could potentially be used to tune permeability.

Zeolite H-Beta was identified as a candidate catalyst for encapsulation because of its very localized acidic activity. Specifically, since its protons are tightly held on the negatively charged framework, any reaction with encapsulated zeolite should occur entirely within the capsule. It was envisaged that the capsule could be used thus to protect to an enzyme in the same system from reacting with the zeolite while allowing substrate and product to permeate and react as normal.

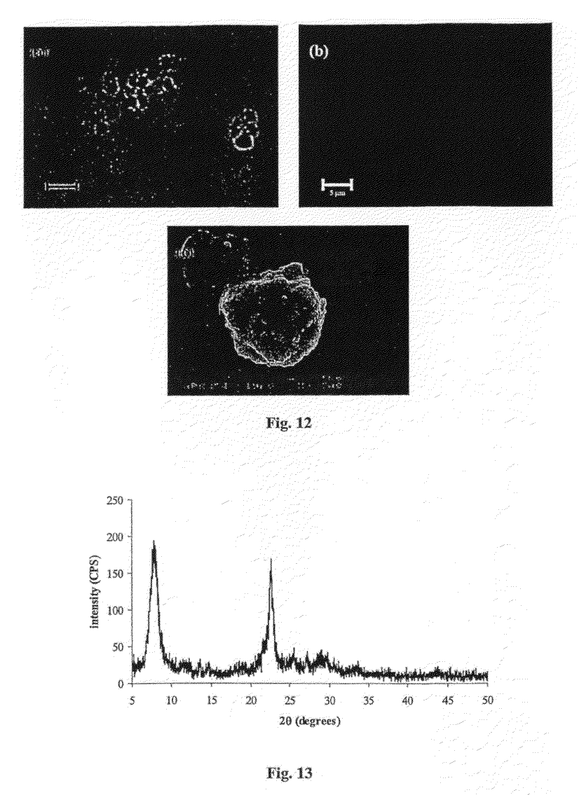

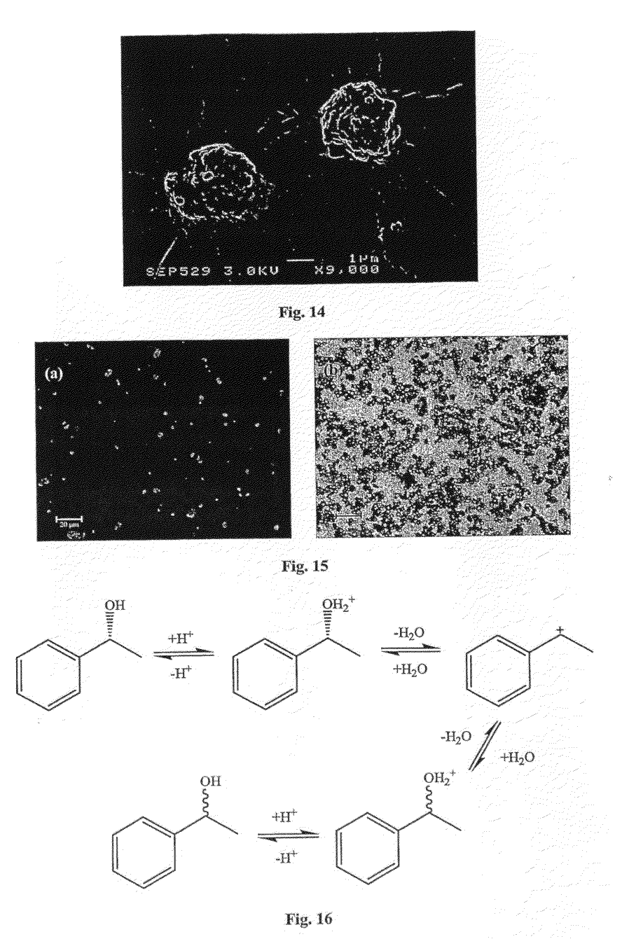

Zeolite Beta was prepared using Aerosil 200 as the silica source and tetraethylammonium hydroxide as the template. The X-Ray Diffraction pattern possessed peaks at 2θ 7.8 and 22.58° that are diagnostic of Zeolite Beta (see FIG. 13). Upon examination with SEM, the zeolite particles were found to be roughly spherical in shape and about 200 nm in diameter (FIG. 14). FIG. 14 shows SEM of Zeolite Beta, showing aggregates of crystals with average diameter about 200 nm.

The acidic form, Zeolite H-Beta, was obtained by ion-exchange of the zeolite with an aqueous ammonium nitrate solution, followed by overnight calcination in air at 550° C.

While the zeolite itself could be imaged easily using SEM techniques, encapsulation of the zeolite was more difficult to assay. SEM is not capable of imaging the interior of capsules, and conventional transmitted light microscopy would involve significant difficulties in resolving the submicron zeolite particles. However, given the fluorescent properties of the polyelectrolyte capsules already synthesized, epifluorescence microscopy appeared a good assay to determine encapsulation of the zeolite. In this way, ordinarily irresolvable zeolite particles could be tagged with a strongly luminescent fluorophore and thus imaged within their capsules. Furthermore, as Zeolite Beta has a negatively charged framework that requires cations to achieve charge balance, fluorescence-tagging could be achieved easily by ion-exchange of the counterions with a suitable fluorescent cation. The metal complex [Ru(bipy)₃]²⁺ was chosen because it is a well-known fluorophore and its luminescence properties have been extensively studied. Ion-exchange of Zeolite H-Beta with an aqueous solution of [Ru(bipy)₃]Cl₂ yielded, after drying, peach-coloured, fluorescent zeolite particles that were easily identifiable using fluorescence microscopy.

Surface precipitation of polyelectrolytes from aqueous solution (LbL assembly) provided an ideal means of encapsulating zeolite particles. This was first attempted by embedding zeolite particles in calcium carbonate core templates followed by polyelectrolyte coating and dissolution of the template, leaving “free” zeolite particles inside otherwise hollow capsules. Zeolite-containing calcium carbonate templates were prepared by slow addition of aqueous sodium carbonate and calcium chloride solutions to a suspension of zeolite in water with rapid stirring. This process yielded amorphous, spherical calcium carbonate particles of diameter 8-10 μm (determined by light microscopy). When fluorescence-tagged zeolite was used, epifluorescence microscopy to revealed that the individual zeolite particles did not remain free in suspension but were in fact embedded in the calcium carbonate templates, as shown in FIG. 15. FIG. 15 shows micrographs (×40 magnification) of calcium carbonate templates containing fluorescence-tagged Zeolite Beta: (a) epifluorescence microscopy image (λₑₓ 450-490 nm); and (b) light microscopy image of the same field, showing that the regions where fluorescence was observed are occupied by calcium carbonate templates. This demonstrates the embedding of the zeolite particles in the templates.

Subsequent encapsulation of these templates and dissolution of the calcium carbonate with aqueous EDTA solution yielded encapsulated zeolite nanoreactors. As shown in the epifluorescence micrograph (FIG. 8), the orange fluorescence of the tagged zeolite was only observed within the blue fluorescence of the capsule, thus demonstrating successful encapsulation of the zeolite.

Zeolite was also encapsulated by direct PE coating of its surface without the use of calcium carbonate. This procedure had the advantage that it avoided the extra steps associated with the precipitation and subsequent dissolution of calcium carbonate. Encapsulation of the zeolite particles by this method was confirmed by comparison of the transmitted light microscopy image (showing the zeolite particles (FIG. 7(a))) with the epifluorescence microscopy image (showing the fluorescent coating (FIG. 7(b))). This coating method eliminated the need for fluorescence-tagging as encapsulation could be assayed by the concomitance of the fluorescent coating in the epifluorescence image with the zeolite particles in the transmitted light microscopy image. Two different kinds of polyelectrolyte solutions were used: those with 0.5 M sodium chloride additive and those without. Both methods gave effective encapsulation.

The catalytic activity of all forms of coated zeolite nanoreactor was tested by observing the rate at which they racemized (R)-1-phenylethanol. As can be seen in FIG. 9, the free zeolite racemized the substrate the fastest, while the zeolite encapsulated with polyelectrolyte solutions with no sodium chloride additive was slightly slower. This small diminution in the reaction rate was presumably due to the limitations of substrate diffusion through the capsule membrane. Similar decreases in catalytic activity attributed to substrate diffusion through the PE capsule membranes have been reported for other applications.

The zeolite coated with polyelectrolyte solutions containing 0.5 M sodium chloride was much less active, though it did racemize the substrate eventually (thus 74% ee after 18 h, 9% ee after 3 days). This decrease in catalytic activity was attributed to to ion-exchange of the acid sites with the Na⁺ ions in the polyelectrolyte solutions during the coating process. The zeolite nanoreactors prepared via calcium carbonate templates, however, did not exhibit any racemization even after 1 day. This lack of activity was attributed to quenching of the zeolite's acidity by the basic calcium carbonate template during precipitation. The inventors hypothesize that the loss of activity for both kinds of nanoreactor could be reversed by ion-exchange with dilute acid solution, providing both a test of the mechanism for loss of activity in these processes and a means of catalyst reactivation. Finally, it should be noted that the empty nanocapsules themselves demonstrated no racemization of the substrate.

Since the zeolite particles coated without the sodium chloride additive exhibited the best catalytic activity among the encapsulated samples, only zeolites coated in this manner were employed as nanoreactors in all later reactions.

There are many potential means of racemization of chiral secondary alcohols. FIG. 16 shows a mechanism of acid-catalyzed racemization of (R)-1-phenylethanol. Under acidic conditions, chiral alcohols may be racemized by protonation of the hydroxyl group, loss of water, and formation of a prochiral, planar sp² carbenium ion. Addition of water thereupon is unselective and thus produces a racemic mixture (FIG. 16). The mechanism for racemization by acidic zeolites, such as Zeolite H-Beta, has been proven to be catalyzed by Brönsted, not Lewis, acidity, as shown in FIG. 16. Indeed, the production of the carbenium intermediate was further evidenced by the colour change of the zeolite in the presence of the substrates, orange in the case of 1-phenylethanol and pink in the case of 1-indanol. Similar colour changes have been attributed to the formation of the relevant carbenium ion on the zeolite. Furthermore, the zeolite's negatively charged framework and pores of molecular dimensions (about 6 Å) are excellent hosts for carbenium ions. This property makes zeolites an excellent medium for the generation and stabilization of carbenium ions. These characteristics are likely to contribute to the efficacy of Zeolite H-Beta as a racemization catalyst.

The formation and stabilization of carbenium ions in the pores of the zeolite also raised problems. Over long reaction times the zeolite tended to consume all of the substrate, converting it either to carbenium ions or, further still, to the dehydration product (FIG. 17). FIG. 17 illustrates the mechanism for racemization of (R)-1-phenylethanol, showing the dehydration side-reaction to give styrene as a byproduct. Indeed, for the racemization of 1-phenylethanol, an increasing amount of styrene formed over time. This elimination can be suppressed by performing the reaction in a water rich environment. Unfortunately, in a DKR reaction using lipase-catalyzed esterification as the resolution step, an excess of water can also promote the lipase-catalyzed hydrolysis of the products, impairing the esterification reaction. For this reason biphasic systems have been tested as a convenient means of resolving the incompatibility of the enzyme and zeolite in past DKR reactions.

Candida antarctica lipase B (CALB) was supplied as an aqueous preparation (Lipozyme CALB L). This preparation was unsuitable for use due to its immiscibility with most organic solvents (and therefore performed slower reactions in these media) as well as its tendency to deactivate the zeolite catalyst (presumably due to ion-exchange by the preservative salts in the aqueous formulation). Consequently, the enzyme was dialyzed extensively against de-ionized water and lyophilized, yielding the pure enzyme as pale yellow-orange flakes. Lyophilization alone was ineffective due to the presence of cryoprotectants (glycerol and sorbitol) in the aqueous preparation.

To establish the integrity of the lyophilized enzyme its activity was tested in various organic solvents which had earlier been used successfully with CALB, by monitoring the rates of esterification of 1-phenylethanol with vinyl acetate in these solvents. In all cases, the (R)-acetyl ester was selectively formed with >99% enantiomeric excess. Moreover, the selection of the (R)-enantiomer of 1-phenylethanol was in agreement with the general enantioselective preference of lipases documented in the literature, which can largely be predicted from the steric bulk of a substrate's substituents and the stereochemistry of the enzyme active site Commonly, steric size of the substituents governs the preference of the enzymes and, in general, gives lipases and proteases opposite enantio-preferences for a given substrate.

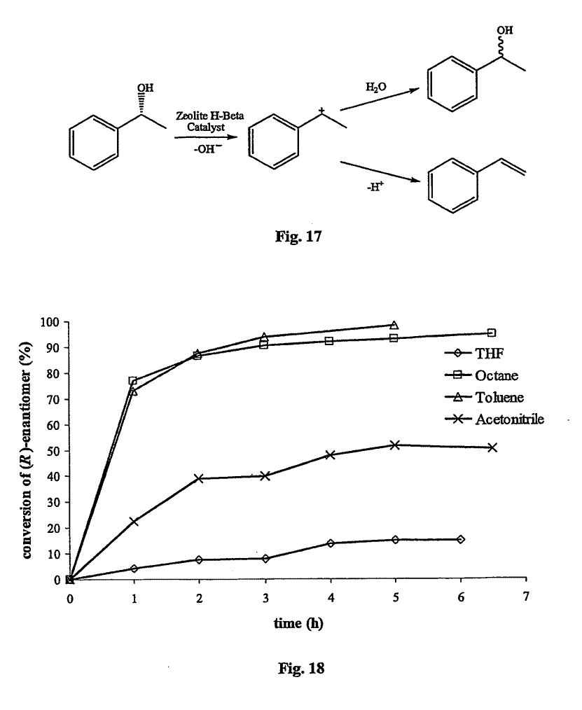

FIG. 18 shows a graph illustrating the progress over time of CALB-catalyzed selective esterification of 1-phenylethanol with vinyl acetate in different solvents. Reaction conditions: racemic 1-phenylethanol (20 μL, 0.165 mmol), vinyl acetate (10 eq, 2 eq/h), dry CALB (10 mg), n-dodecane (internal standard, 20 μL, 88 μmol), solvent (10 mL), air atmosphere, 60° C. As can be seen in FIG. 18, octane and toluene proved to be the most efficient organic solvents tested, while the more polar THF and acetonitrile were less effective. This trend of increasing activity with hydrophobicity is well known, both for CALB and enzymes in general. Water has been hypothesized to form a monolayer around the enzyme that provides a level of lubrication and flexibility that is crucial for catalysis. Hydrophobic solvents, therefore, provide greater activity since the enzyme can more readily abstract water from them.

Having examined the properties of the individual catalysts, encapsulated zeolite nanoreactors and CALB, it remained to test their ability to coexist in a single reaction system. Dynamic Kinetic Resolution was an excellent test reaction for this purpose, as it required both a selective conversion and an in situ racemization of the starting materials. In this case, the Dynamic Kinetic Resolution of secondary alcohols was attempted using encapsulated zeolite as a racemization catalyst and CALB as an enantioselective esterification catalyst (see FIG. 10). These reactions were performed under a range of different conditions, varying solvent, and relative catalyst and substrate concentration (see Table 1). The substrate 1-phenylethanol was chosen since it had been tested previously for ease of both racemization and CALB-catalyzed selective esterification, and also because its enantiomers were known to be well resolved using chiral GC. Control reactions were also employed, using either free (unencapsulated) zeolite or no zeolite in place of the coated zeolite nanoreactors. This was done in order to gauge the effects of zeolite-enzyme interference and the ability of zeolite encapsulation to reduce these effects.

TABLE 1
Results of Dynamic Kinetic Resolution of 1-phenylethanol using Zeolite H-Beta nanoreactors and
the enzyme CALB (10 mg) in air at 60° C.

| | | | Vinyl | | | | | Styrene | (R)-ester | (S)-ester | |
|---|---|---|---|---|---|---|---|---|---|---|---|
| | Substrate | Zeolite | Acetate | | Time | Condition | Conversion | yield | yield | yield | Product ee |
| Entry | (μL) | (mg) | (eq) | Solvent | (h) | of Zeolite^{[a]} | (%) | (%) | (%)^{[b]} | (%)^{[b]} | (%)^{[c]} |
| a | 100 | 10 | 10 | Toluene, | 70 | Coated | 50 | 0 | 45 | 0 | >99 |
| | | | | 50 mL | | Free | 67 | 4 | 53 | 3 | 88 |
| | | | | | | Absent | 52 | 0 | 50 | 0 | >99 |
| b | 100 | 100 | 10 | Toluene, | 46 | Coated | 60 | 18 | 30 | 5 | 73 |
| | | | | 50 mL | | Free | 94 | 36 | 17 | 15 | 8 |
| | | | | | | Absent | 49 | 0 | 51 | 0 | >99 |
| c | 20 | 20 | 10 | Toluene, | 5 | Coated | 100 | 26 | 34 | 17 | 34 |
| | | | (2 eq/h) | 10 mL | | Free | 100 | 34 | 16 | 16 | 1 |
| | | | | | | Absent | 45 | 0 | 46 | 0 | >99 |
| d | 20 | 10 | 10 | Toluene, | 29 | Coated | 100 | 20 | 53 | 12 | 64 |
| | | | (2 eq/h) | 10 mL | | Free | 100 | 38 | 23 | 18 | 11 |
| | | | | | | Absent | 54 | 0 | 47 | 0 | >99 |
| e | 20 | 10 | 10 | Octane, | 22 | Coated | 100 | 10 | 63 | 7 | 81 |
| | | | (2 eq/h) | 10 mL | | Free | 100 | 12 | 52 | 7 | 75 |
| | | | | | | Absent | 51 | 0 | 42 | 0 | >99 |
| f | 20 | 10 | 10 | Octane, | 92 | Coated | 70 | 5 | 53 | 2 | 94 |
| | | | (2 eq/h) | 10 mL | | Free | 76 | 7 | 50 | 2 | 91 |

^{[a]}Refers to form of zeolite used.
^{[b]}Determined using GC calibrated for the products, accurate to within about 1-2%.
^{[c]}Determined using GC.

TABLE 2
Attempted Dynamic Kinetic Resolution using 1-indanol
(10 mg CALB, 3 mg zeolite).

| | Vinyl | | | | | |
|---|---|---|---|---|---|---|
| Substrate | Acetate | | Time | Condition | (R)-ester | Product ee |
| (μL) | (eq) | Solvent | (h) | of Zeolite | yield (%) | (%)^{[c]} |
| 20 | 10 | Octane, | 2 | Coated | 57 | 92 |
| | (2 eq/h) | 10 mL | | Free | 42 | 83 |

As shown in Table 1, the primary features of these reactions were the marked improvements, in both yield and enantioselectivity, obtained when the zeolite catalyst was encapsulated, and markedly worse enantioselectivities obtained when free zeolite was employed compared to no zeolite. These data suggested both that the zeolite interfered significantly with the enzyme's activity and selectivity (by comparing results with free zeolite and no zeolite: see in particular entry d in Table 1) and also that the capsule, when employed, substantially attenuated this interference. This result was unsurprising as enzymes are well known as pH-sensitive catalysts and can exhibit very different activity and selectivity when exposed to an acidic or basic environment, in this case the locally acidic zeolite surface. The semipermeable capsule was expected to exclude the macromolecular enzyme and thereby diminish interference by the zeolite. Moreover, tests showed no significant racemization of the ester product over time, nor did the zeolite catalyze significant achiral esterification on its own, thus confirming that the reduction in ee was due to impairment of the enzyme.

Furthermore, while higher enantiomeric excesses were obtained by using a lower zeolite to enzyme ratio to reduce enzyme impairment further (compare entries c and d in Table 1), too low a concentration of zeolite could have reduced the rate of racemization below what was necessary to allow DKR to proceed effectively (i.e. in >50% yield: see entry a in Table 1). By tuning the relative concentrations of zeolite nanoreactors and CALB, yields of the selected product enantiomer were obtained that unambiguously crossed the 50% barrier of classical kinetic resolution, thus proving the occurrence of Dynamic Kinetic Resolution. Currently, the best result obtained was a 63% yield of the (R)-ester in 81% ee after 22 h (entry e in Table 1).

Nevertheless, significant loss of the desired product from side reactions remained evident. While conversion was often quantitative (entries c-e in Table 1), large amounts of styrene were formed (up to 26% when encapsulated zeolite was used and up to 38% for free zeolite). Moreover, overall yields of ester were limited to 65-70% at best, further illustrating the importance of the dehydration side-reaction. Even taking into account the dehydration side-reaction, a substantial amount of reagent remained unaccounted for (e.g. 34% for entry c using free zeolite in Table 1), much of which was suspected to be sequestered in the pores of the zeolite as the stabilized carbenium ion.

One potential solution to the losses to these side-reactions is the addition of very small quantities of water (below the saturation level of the solvent) to the system. This may not only lead to a reduction in the rate of the elimination reaction, but may also significantly increase the rate of enzyme-catalyzed esterification. By adding small enough quantities of water it is envisaged that the improvement in enzyme activity could outweigh the concomitant increase in the rate of ester hydrolysis.

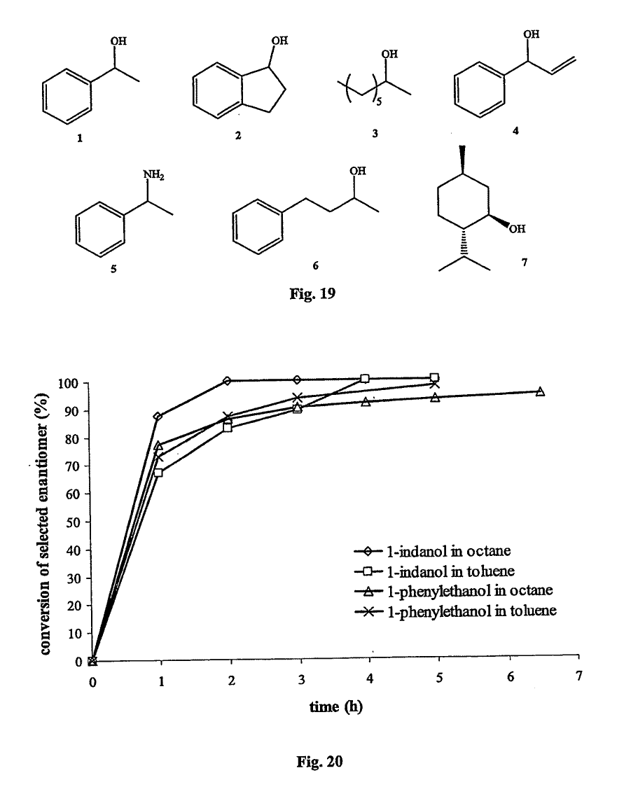

Since 1-phenylethanol was observed to undergo successful Dynamic Kinetic Resolution, several other substrates were used to test the generality of the protocol. Indeed, other Dynamic Kinetic Resolution reactions employing the enzyme CALB have been proven successful for a wide variety of substrates. Some of the successful substrates are shown in FIG. 19.

Substrates 2-5 (FIG. 19) were tested as candidates for the present DKR protocol. All four were found to react with the enzyme alone but with some limitations. Substrate 4, α-vinylbenzyl alcohol, underwent esterification very slowly, giving only 73% conversion of the selected enantiomer (i.e. 37% of the racemic mixture) after 48 h. Substrate 5,1-phenylethylamine, was consumed in both enantiomeric forms after only 1 h, but gave multiple products, including racemic 1-phenylethyl acetamide, contrary to expectations of enantioselectivity. Moreover, 2-octanol (3), while selectively esterified reasonably quickly (88% conversion of the selected enantiomer after 24 h, with product in >99% ee), showed no racemization when reacted with Zeolite H-Beta for 1 day. The racemization activity of (−)-menthol (7) was also tested but it too showed no racemization with Zeolite H-Beta after 1 day.

In contrast, 1-indanol (2) proved a much more promising substrate. Tests of the rate of CALB-catalyzed esterification in octane and toluene showed that 1-indanol was esterified very quickly and selectively (>99% ee) in both solvents, and even faster than 1-phenylethanol in octane, as shown in FIG. 20. FIG. 20 shows a graph illustrating progress over time of CALB-catalyzed selective esterification of 1-indanol with vinyl acetate in different solvents and comparison with that of 1-phenylethanol. Reaction conditions were: racemic substrate (0.165 mmol), vinyl acetate (153 μL, 1.65 mmol, 2 eq/h), dry CALB (10 mg), n-dodecane (internal standard, 20 μL, 88 μmol), solvent (10 mL), air atmosphere, 60° C.

The addition of zeolite to enantio-enriched 1-indanol gave very fast racemization and consumption of the substrate, and the zeolite changed to bright pink in colour due to production of the carbenium ion. Formation of the dehydration product, indene, was also observed in the gas chromatograph.

Given the activity of the individual catalysts towards 1-indanol, a DKR reaction was attempted using the encapsulated zeolite nanoreactors and dry CALB in octane. Using 3 mg of coated zeolite with 10 mg of CALB and otherwise standard reaction conditions, a 57% yield of the selected enantiomer of 1-indanyl acetate was obtained in 92% ee after only 2 h. As observed for 1-phenylethanol, the control reaction with uncoated zeolite performed significantly worse, reaching only 42% yield of the selected enantiomer of 1-indanyl acetate in 83% ee after 2 h, again confirming the protective capabilities of the nanocapsules.

It seems, therefore, that while zeolites are very active catalysts for the racemization of benzylic alcohols such as 1-indanol and 1-phenylethanol, they are inactive towards aliphatic alcohols such as 2-octanol and menthol. This phenomenon is presumably due to to the relative stabilities of the carbenium intermediates formed in the process of racemization (see FIG. 16) which are resonance stabilized for benzylic, but not aliphatic, alcohols. As such, Zeolite H-Beta racemization (and therefore the present DKR protocol) is best suited to substrates that possess a resonance stabilizing α-substituent. Nevertheless, the protocol has been shown to produce successfully >50% yields of selected enantiomers of some substrates in moderate to good ee. This is the first example of catalyst protection using nanoreactors in Dynamic Kinetic Resolution to date and also represents a new method of catalyst protection for multi-catalytic systems.

### Experimental Details

The following reagents were used as received: aqueous tetraethylammonium hydroxide (35 wt. %), hypophosphorous acid (50 wt. %), poly(diallyldimethylammonium chloride) (MW 100-200 kDa, 20 wt. % in water), poly(sodium 4-styrenesulfonate) (MW 70 kDa), α-methylbenzyl acetate, α-vinylbenzyl alcohol, L-menthol (Sigma-Aldrich); sodium chloride, ammonium nitrate, ethylenediaminetetraacetic acid disodium salt, toluene, pyridine (Ajax); potassium chloride, 1-phenylethanol, acetyl chloride, 2,2′-bipyridine, acetone (Merck); amorphous silica (Aerosil 200) (Degussa); sodium hydroxide (APS); sodium aluminate (50-56 wt. % Al₂O₃, 40-45 wt. % Na₂O) (Riedel-de Haën); ruthenium(III) chloride hydrate, palladium(II) chloride (Strem); calcium chloride dihydrate, sodium carbonate (AnalaR); n-dodecane (BDH); hexane, ethyl acetate (Redox); 1-indanol, (R)-(+)-1-phenylethanol, 1-phenylethylamine (Alfa-Aesar); 2-octanol, indene (Fluke). Lipozyme Candida antarctica Lipase B (1-10 wt. % in aqueous formulation) was kindly donated by Novozymes, and was dialyzed extensively against de-ionized water using PROGEN SnakeSkin Pleated Dialysis Tubing (10,000 MWCO, 22 mm) and lyophilized with a freeze drier prior to use. Chromatography was carried out with Ajax silica gel (230-400 mesh) for flash columns, and preparative thin-layer chromatography (TLC) was conducted on Merck plates. Unless otherwise specified, solvents were removed with a rotary evaporator equipped with a diaphragm pump and dynamic pressure regulator. Toluene and diethyl ether were deoxygenated and dried over activated alumina using an apparatus modified from that described in the literature.

¹H NMR (300.13 MHz) and ¹³C{¹H} NMR (75.48 MHz) spectra were recorded with a Bruker DPX300 spectrometer at 300 K and were referenced internally to residual solvent. Mass spectra were obtained using either a Finnigan LCQ ion trap spectrometer (ESI) or a Finnigan Polaris Q ion trap mass spectrometer operating at 70 eV ionization energy, with a Trace GC having a 15 m ZB-5 column, 5% phenyl 95% dimethylpolysiloxane (GC/MS). Field Emission Scanning Electron Micrographs were obtained using a JSM-6000F Scanning Electron Microscope operating at 3.0 kV. X-Ray Diffraction patterns were recorded using a Siemens D5000 X-Ray Diffractometer equipped with a liquid nitrogen cooled germanium solid-state detector using Cu Kα radiation at 40 kV. Transmitted Light Microscopy (LM) and Epifluorescence Microscopy (FM) were performed using a Nikon Eclipse E800 fluorescence microscope fitted with Nomarski DIC optics and Nikon Plan Fluor×10 (NA 0.30, dry), ×20 (NA 0.50, dry), ×40 (NA 0.75, dry) and ×100 (NA 1.30, oil) objectives. The DAPI filter set (BP330-380, DIC400, LP420) was employed for observation of PE-capsule and [Ru(bipy)₃]²⁺ fluorescence. Images were captured with a PCO Sensicam 12-bit cooled imaging camera. Samples were mounted on slides in either glycerol or water and covered with glass coverslips. Gas chromatography was performed using a Hewlett-Packard 5890A gas chromatograph fitted with a VarianCP-Chirasil-Dex CB column (25 m×0.32 mm I.D.; 0.25 μm film thickness), a split/splitless inlet, and an FID detector. Data were collected and analyzed using GC ChemStation software. UV-Visible spectra were recorded using a Varian Cary 1E UV-Visible spectrophotometer.

Zeolite H-Beta was prepared with batch composition 1.97 Na₂O:1.00 K₂O :12.5(TEA)₂O:Al₂O₃:50 SiO₂:750H₂O:2.9HCl. A polypropylene container was charged with distilled water (4.66 mL, 259 mmol), aqueous tetraethylammonium hydroxide (35 wt. %, 10.40 g, 71 mmol), sodium chloride (52 mg, 0.91 mmol) and potassium chloride (144 mg, 1.96 mmol). The mixture was stirred until a clear solution formed. Amorphous silica (2.93 g, 48.9 mmol) was added and the mixture was stirred for 1 h to give a clear solution. A solution of sodium hydroxide (33 mg, 0.83 mmol) and sodium aluminate (179 mg, ˜0.93 mmol Al₂O₃, ˜1.2 mmol Na₂O) in distilled water (2.00 mL, 111 mmol) was added and the mixture stirred vigorously for 1 h to give a homogeneous gel. The gel was heated in a Teflon-lined stainless-steel autoclave at 135° C. for 20 h. After cooling, the reaction mixture was centrifuged (4000 rpm, 20 min) and washed with distilled water (3×10 mL). The zeolite crystals were dried overnight at 120° C. to form a white, solid, microcrystalline layer. The zeolite was calcined in air at 550° C. for 6 h after a ramp rate of 1° C./min, and subsequently suspended for 20 min in aqueous 1 M ammonium nitrate (3×90 mL), washed with distilled water (3×30 mL) and dried at 80° C. to give the NH₄-Beta zeolite. This product was further calcined overnight at 550° C., yielding the acidic Zeolite H-Beta as a fine, white, crystalline powder (1.11 g, ˜45% yield based on Al): XRD 2θ(Iᵣₑₗ) 7.82 (100), 22.58° (87) (c.f. lit. 7.69, 22.4°.

Fluorescent [Ru(bipy)₃]Cl₂.6H₂O was prepared using literature methods (J. A. Broomhead, C. G. Young, Inorg. Synth. 1982, 21, 127-128). Thus RuCl₃.xH₂O (0.5 g) was pre-dried at 120° C. for 3 h, ground, and heated at 120° C. for a further 1 h. The dried RuCl₃ (431 mg, 2.08 mmol) and 2,2′-bipyridine (0.97 g, 6.2 mmol) were then added to de-ionized water (40 mL). Freshly prepared sodium phosphinate solution (2 mL, ±40 wt. %) was added and the mixture was heated at reflux for 40 min, during which time the solution changed colour from green to brown and then to orange. The reaction mixture was filtered to remove undissolved material. Potassium chloride (13.6 g 0.184 mol) was added, precipitating the crude product as a red-orange solid. The mixture was then heated at reflux for 1 h to give a deep red solution, which, upon cooling to room temperature, yielded red crystals. The crystals were removed by filtration and washed with ice-cold aqueous acetone (2×4 mL, 8% v/v) and acetone (30 mL), followed by drying in air, affording the product as shiny, red, platelike crystals (844 mg, 54%): ¹H NMR (CD₃OD, ppm) δ 7.19 (d, 6H), 6.61 (td, 6H), 6.31 (d, 6H), 5.97 (td, 6H); MS m/z (%) 285.2 (100) [M]²⁺ (calculated 285.1), 157.1 (8) [bipyridine+1]⁺ (calculated 157.1); UV-Vis (EtOH, m) λₘₐₓ 449 (ε=9000 M⁻¹ cm⁻¹).

Zeolite H-Beta (99 mg) underwent ion-exchange with an aqueous solution of [Ru(bipy)₃]Cl₂ (3×1 mL, 14 mM). The zeolite was centrifuged (13400 rpm, 5 min), washed with distilled water (3×1 mL) and subsequently dried at 120° C. for 4 h, yielding a fine peach-coloured powder (91 mg).

Zeolite H-Beta (100 mg) was impregnated with an aqueous solution of PdCl₂ (140 μL, 0.14 M) and left for 20 min. The zeolite was dried at 110° C. for 3 h and subsequently calcined at 300° C. for 4 h, yielding the Pd-loaded zeolite (2 wt. %) as a grey powder (104 mg).

Core templates were prepared as follows. Aqueous CaCl₂ solution (20 mL, 1 M) was diluted with de-ionized water (160 mL). Aqueous Na₂CO₃ solution (20 mL, 1 M) was rapidly added to the vigorously stirred solution, resulting in the formation of a white suspension. The suspension was centrifuged (2000 rpm, 10 min), and the solid washed with distilled water (3×70 mL) and acetone (50 mL). The solid CaCO₃ was then resuspended in acetone (50 mL) and dried at 60° C., yielding the templates as a fine white powder (1.34 g) with a particle size of 5-10 μm (determined by light microscopy).

Polyelectrolyte capsules were prepared as follows. Calcium carbonate core templates prepared as described earlier were coated with polyelectrolyte using the Layer-by-Layer (LbL) method. Poly(diallyldimethylammonium chloride) (PDA, MW 100-200 kDa) was deposited to form the positive layers from an aqueous solution (4 g L⁻¹ PDA, 0.5 M NaCl). Poly(sodium 4-styrenesulfonate) (PSS, MW 70 kDa) was deposited to form the negative layers from an aqueous solution (5 g L⁻¹ PSS, 0.5 M NaCl). The solutions, starting with PDA, were alternately added in 300-μL portions to CaCO₃ templates (99 mg) to form multilayers, leaving the mixture for 20 minutes each time, and centrifuging (13,400 rpm, 90 s) and washing with water (3×1 mL) between additions. After addition of 3 PE bilayers, the CaCO₃ cores were removed by washing with an EDTA solution (3×1 mL, 0.2 M, pH 7) and the capsules were incubated for 10 h in that solution. The capsules were subsequently centrifuged (13,400 rpm, 5 min), washed with water (1 mL) and ethanol (3×1 mL), and stored in ethanol.

Zeolite H-Beta (215 mg) was ground and suspended in de-ionized water (−5 mL). The mixture was sonicated to break up crystal aggregates, yielding a cloudy, white suspension. The zeolite suspension was diluted with de-ionized water (1.8 L), and aqueous solutions of Na₂CO₃ (0.40 M, 100 mL) and CaCl₂.2H₂O (0.40 M, 100 mL) were added very slowly (˜1 drop/5 s) to the vigorously stirred suspension via addition funnels. A white suspension formed and the supernatant was decanted. The CaCO₃ templates were washed (3×H₂O and 1×acetone), resuspended in acetone, and dried in vacuo at 50° C., affording the zeolite-containing templates as a fine white powder (3.14 g). Light microscopy revealed mainly spherical particles, 1-5 μm in diameter. The templates were encapsulated and dissolved using the protocol described in Section 2.4.2, yielding encapsulated zeolite.

Zeolite H-Beta (25 mg) was directly coated with PDA and PSS solutions as described earlier. The coating process was also conducted using PDA and PSS solutions without added sodium chloride. In either case, the coated zeolite was washed with water (1 mL) and ethanol (3×1 mL), and resuspended in ethanol for storage.

The typical reaction conditions for racemization of 1-phenylethanol were as follows: toluene (50 mL) was added to free or encapsulated Zeolite H-Beta catalyst (10 mg, prepared as described earlier), or an approximately equal number of empty nanocapsules (prepared as described earlier) and stirred. The substrate, (R)-1-phenylethanol (100 μL, 0.827 mmol), and n-dodecane (internal standard, 100 μL, 0.439 mmol) were added and the reaction mixture was heated at 60° C. The reaction was monitored using GC and enantiomeric excesses were determined by comparing the to integrated areas of peak signals in the gas chromatograph.

Unless stated otherwise, typical reaction conditions for enzyme-catalyzed selective esterification were as follows: dry lipase (10 mg) was added to the solvent (10 mL) and the mixture briefly sonicated to dissolve the enzyme. To the magnetically stirred solution were added the substrate (0.165 mmol) and n-dodecane (internal standard, 20 μL, 88 μmol). The reaction mixture was heated at 60° C., with vinyl acetate (153 μL, 1.65 mmol) introduced at a rate of 2 eq/h. The reaction was monitored by GC and enantiomeric excesses were determined by comparing the integrated areas of peak signals in the gas chromatograph. Yields were determined by GC after calibration with the substrate and products.

1-indanyl acetate was prepared for calibration as follows. Dry diethyl ether (30 mL), 1-indanol (202 mg, 1.51 mmol), and pyridine (241 μL, 2.98 mmol) were combined. Acetyl chloride (1.06 mL, 14.9 mmol) was added to the solution and a white precipitate formed. The mixture was stirred overnight at room temperature, filtered, and the solvent, excess pyridine, and acetyl chloride were removed from the filtrate in vacuo. The residue was purified by column chromatography (1:8 ethyl acetate/hexane), yielding the product (72 mg, 27%) as a very pale yellow oil: ¹H NMR (CD₃OD, ppm) δ 7.35 (d, 1H), 7.26 (m, 2H), 7.20 (m, 1H), 6.14 (dd, 1H), 2.96 (m, 1H), 2.75 (m, 1H), 2.45 (m, 1H), 2.06 (m, 1H), 2.02 (s, 3H); ¹³C{¹H} NMR (CD₃CN, ppm) δ 171.7, 145.5, 142.3, 129.8, 127.5, 126.3, 125.8, 79.0, 33.0, 30.7, 21.4.

Unless stated otherwise, typical reaction conditions for DKR reactions were as follows: dry CALB enzyme (10 mg) was added to the solvent (10 mL) and the mixture sonicated briefly to dissolve the enzyme. The substrate (0.165 mmol), n-dodecane (internal standard, 20 μL, 88 μmol) and PE-coated Zeolite H-Beta nanoreactors (10 mg) were added. The reaction mixture was heated at 60° C., with vinyl acetate (153 μL, 10 eq) introduced at a rate of 2 eq/h. The reaction was monitored by GC and enantiomeric excesses were determined by comparing the integrated areas of peak signals in the gas chromatograph. Yields were determined by GC after calibration with the substrate and products.

Typical reaction conditions for the racemization of (R)-1-phenylethanol by zeolite catalysts were as follows: (R)-1-phenylethanol (100 μL, 0.827 mmol), zeolite catalyst (10 mg), n-dodecane (internal standard, 100 μL, 0.439 mmol), and toluene (50 mL) were combined and heated at 60° C. in air. Typical reaction conditions for the DKR of secondary alcohols were as follows: dry CALB enzyme (10 mg) was added to the solvent (toluene or octane, 10 mL) and the mixture sonicated briefly to dissolve the enzyme. The substrate (0.165 mmol), n-dodecane (internal standard, 20 μL, 88 μmol), and PE-coated Zeolite H-Beta nanoreactors (10 mg) were added. The reaction mixture was heated at 60° C. in air, with vinyl acetate (153 μL, 10 eq) introduced at a rate of 2 eq/h. Both reactions were monitored by GC (VarianCP-Chirasil-Dex CB column), and enantiomeric is excesses were determined by comparing the integrated areas of peak signals in the gas chromatograph. Yields were determined by GC after calibration with the substrate and products.

1. A reaction system comprising:
a first reaction promoter capable of converting a first substrate into a first substance; and
a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant;
whereby either (a) the first substance is, or is capable of being converted into, the second substrate and, in operation, the conversion of the second substrate by the second reaction promoter occurs to a greater extent than the conversion of the first substrate by the second reaction promoter and the conversion of the second substance by the first reaction is promoter is low, or (b) the second substance is, or is capable of being converted into, the first substrate and, in operation, the conversion of the first substrate by the first reaction promoter occurs to a greater extent than the conversion of the second substrate by the first reaction promoter and the conversion of the first substance in the microcapsules is low.

2. The reaction system of claim 1 wherein, in case (a), the first reaction promoter is capable of interconverting the first substrate and the second substrate, and in case (b), the second reaction promoter is capable of interconverting the first substrate and the second substrate.

3. The reaction system of claim 1 wherein the first and second reaction promoters are catalysts.

4. The reaction system of claim 1 comprising one or more further reaction promoters which are capable of converting the second substance (in case a) or converting the first substance (in case b).

5. The reaction system of claim 1 wherein the first substance (case b) or the second substance (case a) passes slowly through the encapsulant or is incapable of passing therethrough.

6. The reaction system of claim 1 additionally comprising a separator for separating a product from the first reaction promoter and from the microcapsules.

7. The reaction system of claim 6 also comprising a purifier for purifying the separated product.

8. The reaction system of claim 1 wherein the first reaction promoter is a selective reaction promoter.

9. The reaction system of claim 1 wherein the microcapsules are capable of converting the second substrate to the second substance selectively.

10. The reaction system of claim 1 wherein the encapsulant comprises a polymer.

11. The reaction system of claim 1 wherein the encapsulant to comprises at least one positively charged polymeric layer and at least one negatively charged polymeric layer.

12. The reaction system of claim 1 wherein the first and second reaction promoters are capable of interacting so as to deactivate one or both thereof, but in the reaction system of the present invention are at least partially prevented from doing so due to encapsulation of the second reaction promoter.

13. The reaction system of claim 1 wherein first reaction promoter and the microcapsules are distributed within a reaction medium.

14. The reaction system of claim 1 wherein the microcapsules have a mean diameter of between about 0.2 and about 10 microns.

15. The reaction system of claim 1 wherein the microcapsules comprise an energy absorber for absorbing energy in order to promote the reaction promoted by the second reaction promoter.

16. A method for conducting a reaction comprising:
providing a reaction system comprising (i) a first reaction promoter capable of converting a first substrate into a first substance; and (ii) a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant; and
adding either the first substrate or the second substrate or both the first substrate and the second substrate to the reaction system;
whereby either (a) the first substance is, or is capable of being converted into, the second substrate, such that the first substrate is converted either directly or indirectly to the second substrate and the second substrate is converted to the second substance, or (b) the second substance is, or is capable of being converted into, the first substrate, such that the second substrate is converted either directly or indirectly to the first substrate and the first substrate is converted to the first substance.

17. The method of claim 16 whereby in case (a) the first reaction promoter interconverts the first and second substrates and in case (b) the second reaction promoter interconverts the first and second substrates.

18. The method of claim 16 wherein first substrate and the second substrate are added together to the reaction system.

19. The method of claim 16 comprising separating a product from the reaction system.

20. The method of claim 16 comprising heating the reaction system and/or irradiating the reaction system with radiation of a wavelength capable of being absorbed by a component of the reaction system, by the first or second substrate or by more than one of these.

21. A reaction system for Dynamic Kinetic Resolution of a racemic alcohol comprising:
a first catalyst capable of esterifying a first optical isomer of the alcohol to form a first optical isomer of an ester of the alcohol at a rate greater than it esterifies a second optical isomer of the alcohol; and
a plurality of microcapsules, each of said microcapsules comprising a second catalyst encapsulated within an encapsulant, said second catalyst being capable of racemising the second optical isomer of the alcohol, wherein the second optical isomer of the alcohol is capable of passing through the encapsulant to contact the second catalyst and the first optical isomer of the alcohol is capable of passing out of the microcapsules through the encapsulant;
whereby the rate of racemisation of the first optical isomer of the ester of the alcohol in 30 the microcapsules is low.

22. A method for Dynamic Kinetic Resolution of a racemic alcohol comprising:
providing a reaction system comprising (i′″) a first catalyst capable of esterifying a first optical isomer of the alcohol to form a first optical isomer of an ester of the alcohol at a rate greater than it esterifies a second optical isomer of the alcohol; and (ii′″) a plurality of microcapsules, each of said microcapsules comprising a second catalyst encapsulated within an encapsulant, said second catalyst being capable of racernising the second optical isomer of the alcohol, wherein the second optical isomer of the alcohol is capable of passing through the encapsulant to contact the second catalyst and the first optical isomer of the alcohol is capable of passing out of the microcapsules through the encapsulant; whereby the rate of racemisation of the first optical isomer of the ester of the alcohol in the microcapsules is low; and
adding the racemic alcohol to the reaction system;
whereby the first optical isomer of the alcohol is converted to the first optical isomer of the ester and the second optical isomer is racemised to form a mixture of the first and second optical isomers of the alcohol.

23. A product produced by the method of claim 16.

24. A reaction system comprising:
a first reaction promoter capable of converting a first substrate into a first substance; and
a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant;
whereby the first and second reaction promoters are capable of interacting so as to deactivate one or both thereof, but in the reaction system are at least partially prevented from doing so due to encapsulation of the second reaction promoter.

25. The reaction system of claim 24 whereby either (a) the first substance is the second substrate, or (b) the second substance is the first substrate.

26. A method for conducting a reaction comprising:
providing a reaction system comprising (i) a first reaction promoter capable of converting a first substrate into a first substance; and (ii) a plurality of microcapsules, each of said microcapsules comprising a second reaction promoter encapsulated within an encapsulant, said second reaction promoter being capable of converting a second substrate into a second substance, wherein the second substrate is capable of passing through the encapsulant to contact the second reaction promoter and the second substance is capable of passing out of the microcapsules through the encapsulant; and
adding either the first substrate or the second substrate or both the first substrate and the second substrate to the reaction system;
whereby either (a) the first substance is the second substrate, or (b) the second substance is the first substrate, and whereby the first and second reaction promoters are capable of interacting so as to deactivate one or both thereof, but in the reaction system are at least partially prevented from doing so due to encapsulation of the second reaction promoter.

27. A product produced by the method of claim 26.
